SEQUENCE LISTING (70 sequences)

SEQ ID NO: 1 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 2 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 3 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 4 - protein, 194 aa, Mus musculus
MSLLLLGWLPTGMTHQDPPSWGKPRSHRTLRCYSAEELSHGQAPPHLLTRSARWEQALPVALVASLEATGHRRQHEGPLAGTQCPVLRPEEVLEADTHERSISPWRYRIDTDENRYPQKLAVAECLCRGCINAKTGRETAALNSVQLLQSLLVLRRQPCSRDGTADPTPGSFAFHTEFIRVPVGCTCVLPRSTQ

SEQ ID NO: 5 - protein, 197 aa, Macaca fascicularis
MTLLPGLLFLTWLHACLAHQDPFLRGHPHTHGTPRCYSAEELPLGQAPPHLLARGAKWGQALPVALVSSLEAAGHRRRHDRPSAATQCPVLRPEEVLEADTHQRSISPWRYRVDTDEDRYPQKLAFAECLCRGCIDPRTGRETAALNSVRLLQSLLVLRRRPCSRDGSGLPTPGAFAFHTEFIRVPVGCTCVLPRSV

SEQ ID NO: 6 - protein, 637 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDVSHRHLGLLILALLALTALVGVVLVLLGRRLLPGSGRTRPVLLLHAADSEAQRRLVGALAELLRTALGGGRDVIVDLWEGTHVARIGPLPWLWAARERVAREQGTVLLLWNCAGPSTACSGDPQAASLRTLLCAAPRPLLLAYFSRLCAKGDIPRPLRALPRYRLLRDLPRLLRALDAQPATLASSWSHLGAKRCLKNRLEQCHLLELEAAKDDYQGSTNSPCGFSCL

SEQ ID NO: 7 - protein, 5 aa, Artificial Sequence
  source: /note=“Description of Artificial Sequence: Synthetic peptide”
DYAMH

SEQ ID NO: 8 - protein, 17 aa, Artificial Sequence
  source: /note=“Description of Artificial Sequence: Synthetic peptide”
YIGGVGEGTQYAESVKG

SEQ ID NO: 9 - protein, 11 aa, Artificial Sequence
  source: /note=“Description of Artificial Sequence: Synthetic peptide”
GFAIRYYGFDY

SEQ ID NO: 10 - protein, 7 aa, Artificial Sequence
  source: /note=“Description of Artificial Sequence: Synthetic peptide”
GFTVSDY

SEQ ID NO: 11 - protein, 6 aa, Artificial Sequence
  source: /note=“Description of Artificial Sequence: Synthetic peptide”
GGVGEG

SEQ ID NO: 12 - protein, 11 aa, Artificial Sequence
  source: /note=“Description of Artificial Sequence: Synthetic peptide”
GFAIRYYGFDY

SEQ ID NO: 13 - protein, 11 aa, Artificial Sequence
  source: /note=“Description of Artificial Sequence: Synthetic peptide”
SGDKLGDKYAY

SEQ ID NO: 14 - protein, 7 aa, Artificial Sequence
  source: /note=“Description of Artificial Sequence: Synthetic peptide”
QDSKRPS

SEQ ID NO: 15 - protein, 10 aa, Artificial Sequence
  source: /note=“Description of Artificial Sequence: Synthetic peptide”
QVFTFPLVTT

SEQ ID NO: 16 - protein, 110 aa, Artificial Sequence
  source: /note=“Description of Artificial Sequence: Synthetic polypeptide”
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQ

SEQ ID NO: 17 - protein, 120 aa, Artificial Sequence
  source: /note=“Description of Artificial Sequence: Synthetic polypeptide”
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSS

SEQ ID NO: 18 - DNA, 330 bp, Artificial Sequence
  source: /note=“Description of Artificial Sequence: Synthetic polynucleotide”
agctatgaactgacccagccgccgagcgttagcgttagcccaggccagaccgccagcattacctgtagcggcgacaaactgggcgacaaatacgcctactggtatcagcagaaaccgggccagagcccggtgctggttatctatcaggatagcaaacgcccgagcggcattccagaacgctttagcggcagcaacagcggcaacaccgccaccctgaccattagcggcacccaggccgaagacgaagccgattattactgccaggttttcactttcccgctggttactactgtgtttggcggcggtaccaagctgaccgtgctgggccag

SEQ ID NO: 19 - DNA, 360 bp, Artificial Sequence
  source: /note=“Description of Artificial Sequence: Synthetic polynucleotide”
gaagtgcagctgctggaaagcggtggcggtctggtgcagccaggtggtagcctgcgcctgagctgtgccgcaagcggcttcacagtgtccgactacgcaatgcattgggtgcgccaagcaccaggcaaaggcctggaatgggtgagttacataggtggcgtgggtgaggggacacaatatgcagagagcgtgaaaggtcgctttaccattagtcgcgataacagcaaaaacaccctgtatctgcaaatgaacagcctgcgggcagaagataccgcagtttattattgcgcgcgtggtttcgcaatccgttattatggatttgattattggggccagggcaccctggttactgtctcgagc

SEQ ID NO: 20-32 - skipped (13 entries)

SEQ ID NO: 33 - DNA, 15 bp, Artificial Sequence
  source: /note=“Description of Artificial Sequence: Synthetic oligonucleotide”
gactacgcaatgcat

SEQ ID NO: 34 - DNA, 51 bp, Artificial Sequence
  source: /note=“Description of Artificial Sequence: Synthetic oligonucleotide”
tacataggtggcgtgggtgaggggacacaatatgcagagagcgtgaaaggt

SEQ ID NO: 35 - DNA, 33 bp, Artificial Sequence
  source: /note=“Description of Artificial Sequence: Synthetic oligonucleotide”
ggtttcgcaatccgttattatggatttgattat

SEQ ID NO: 36 - DNA, 21 bp, Artificial Sequence
  source: /note=“Description of Artificial Sequence: Synthetic oligonucleotide”
ggcttcacagtgtccgactac

SEQ ID NO: 37 - DNA, 18 bp, Artificial Sequence
  source: /note=“Description of Artificial Sequence: Synthetic oligonucleotide”
ggtggcgtgggtgagggg

SEQ ID NO: 38 - DNA, 33 bp, Artificial Sequence; identical to SEQ ID NO: 35
  source: /note=“Description of Artificial Sequence: Synthetic oligonucleotide”

SEQ ID NO: 39 - DNA, 33 bp, Artificial Sequence
  source: /note=“Description of Artificial Sequence: Synthetic oligonucleotide”
agcggcgacaaactgggcgacaaatacgcctac

SEQ ID NO: 40 - DNA, 21 bp, Artificial Sequence
  source: /note=“Description of Artificial Sequence: Synthetic oligonucleotide”
caggatagcaaacgcccgagc

SEQ ID NO: 41 - DNA, 30 bp, Artificial Sequence
  source: /note=“Description of Artificial Sequence: Synthetic oligonucleotide”
caggttttcactttcccgctggttactact

SEQ ID NO: 42 - protein, 214 aa, Artificial Sequence
  source: /note=“Description of Artificial Sequence: Synthetic polypeptide”
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 450 aa, Artificial Sequence
  source: /note=“Description of Artificial Sequence: Synthetic polypeptide”
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 642 bp, Artificial Sequence
  source: /note=“Description of Artificial Sequence: Synthetic polynucleotide”
agctatgaactgacccagccgccgagcgttagcgttagcccaggccagaccgccagcattacctgtagcggcgacaaactgggcgacaaatacgcctactggtatcagcagaaaccgggccagagcccggtgctggttatctatcaggatagcaaacgcccgagcggcattccagaacgctttagcggcagcaacagcggcaacaccgccaccctgaccattagcggcacccaggccgaagacgaagccgattattactgccaggttttcactttcccgctggttactactgtgtttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 45 - DNA, 1350 bp, Artificial Sequence
  source: /note=“Description of Artificial Sequence: Synthetic polynucleotide”
gaagtgcagctgctggaaagcggtggcggtctggtgcagccaggtggtagcctgcgcctgagctgtgccgcaagcggcttcacagtgtccgactacgcaatgcattgggtgcgccaagcaccaggcaaaggcctggaatgggtgagttacataggtggcgtgggtgaggggacacaatatgcagagagcgtgaaaggtcgctttaccattagtcgcgataacagcaaaaacaccctgtatctgcaaatgaacagcctgcgggcagaagataccgcagtttattattgcgcgcgtggtttcgcaatccgttattatggatttgattattggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaactgctgggaggcccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 46-57 - skipped (12 entries)

SEQ ID NO: 58 - DNA, 15 bp, Artificial Sequence
  source: /note=“Description of Artificial Sequence: Synthetic oligonucleotide”
gactacgctatgcac

SEQ ID NO: 59 - DNA, 51 bp, Artificial Sequence
  source: /note=“Description of Artificial Sequence: Synthetic oligonucleotide”
tatatcggcggcgtgggcgagggcacccagtacgctgagtctgtgaagggc

SEQ ID NO: 60 - DNA, 33 bp, Artificial Sequence
  source: /note=“Description of Artificial Sequence: Synthetic oligonucleotide”
ggcttcgccatccggtactacggcttcgactac

SEQ ID NO: 61 - DNA, 21 bp, Artificial Sequence
  source: /note=“Description of Artificial Sequence: Synthetic oligonucleotide”
ggcttcaccgtgtccgactac

SEQ ID NO: 62 - DNA, 18 bp, Artificial Sequence
  source: /note=“Description of Artificial Sequence: Synthetic oligonucleotide”
ggcggcgtgggcgagggc

SEQ ID NO: 63 - DNA, 33 bp, Artificial Sequence; identical to SEQ ID NO: 60
  source: /note=“Description of Artificial Sequence: Synthetic oligonucleotide”

SEQ ID NO: 64 - DNA, 33 bp, Artificial Sequence
  source: /note=“Description of Artificial Sequence: Synthetic oligonucleotide”
tccggcgacaagctgggcgataagtacgcctac

SEQ ID NO: 65 - DNA, 21 bp, Artificial Sequence
  source: /note=“Description of Artificial Sequence: Synthetic oligonucleotide”
caggactccaagcggccctcc

SEQ ID NO: 66 - DNA, 30 bp, Artificial Sequence
  source: /note=“Description of Artificial Sequence: Synthetic oligonucleotide”
caggtgttcaccttccccctggtcaccacc

SEQ ID NO: 67 - DNA, 330 bp, Artificial Sequence
  source: /note=“Description of Artificial Sequence: Synthetic polynucleotide”
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccag

SEQ ID NO: 68 - DNA, 360 bp, Artificial Sequence
  source: /note=“Description of Artificial Sequence: Synthetic polynucleotide”
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagc

SEQ ID NO: 69 - DNA, 642 bp, Artificial Sequence
  source: /note=“Description of Artificial Sequence: Synthetic polynucleotide”
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaggtcacacacgagggctccaccgtggaaaagaccgtggcccctaccgagtgctcc

SEQ ID NO: 70 - DNA, 1350 bp, Artificial Sequence
  source: /note=“Description of Artificial Sequence: Synthetic polynucleotide”
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagcgcctccaccaagggcccctccgtgttccctctggccccctccagcaagtccacctctggcggcaccgctgccctgggctgcctggtcaaggactacttccccgagcccgtgaccgtgtcctggaactctggcgccctgacctccggcgtgcacaccttccctgccgtgctgcagtcctccggcctgtactccctgtcctccgtcgtgaccgtgccctccagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggccctgcctgcccccatcgaaaagaccatctccaaggccaagggccagccccgcgagccccaggtgtacacactgccccctagccgggaagagatgaccaagaaccaggtgtccctgacctgtctggtcaagggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

(19) United States
(12) Patent Application Publication
(10) Pub. No.: US 2023/0203149 A1
(43) Pub. Date: Jun. 29, 2023

(54) TREATMENT OF ATOPIC DERMATITIS

(71) Applicants: MORPHOSYS AG, Planegg (DE); GALAPAGOS NV, Mechelen (BE)

(72) Inventors: Stefan Haertle, Planegg (DE); Tim Van Kaem, Mechelen (BE); Frederic Paul Vanhoutte, Mechelen (BE)

(21) Appl. No.: 16/647,775

(22) PCT Filed: Sep. 25, 2018
(86) PCT No.: PCT/EP2018/075869

§ 371 Date: Mar. 16, 2020

(30) Foreign Application Priority Data
Sep. 25, 2017 (EP) 17192955.7

(51) Int. Cl. C07K 16/24; A61P 17/06; A61K 9/00

(52) U.S. Cl. CPC C07K 16/244; A61P 17/06; A61K 9/0019; A61K 2039/545

(57) ABSTRACT

The present invention provides a pharmaceutical composition comprising an antibody for antibody fragment specific for IL-17C, in particular MOR106 for use in the treatment of atopic dermatitis and related conditions wherein the antibody is administered to patients at dosages that are beneficial in a clinical setting.

This patent application is the National Stage of International Application No. PCT/EP2018/075869 filed Sep. 25, 2018, which claims the benefit of priority from EP 17192955.7 filed Sep. 25, 2017, each of which is incorporated herein by reference in its entirety.

### FIELD OF THE INVENTION

The present disclosure relates to the treatment of atopic dermatitis. More specifically, the present invention relates to the administration of an antibody or antibody fragment specific for IL-17C to subjects in need thereof. The antibody or antibody fragment specific for IL-17C, in particular MOR106, is administered to patients suffering from atopic dermatitis (AD), in particular to patients suffering from moderate-to-severe atopic dermatitis according to a specific dosing as disclosed herein.

### BACKGROUND

Atopic dermatitis (AD) is a chronic/relapsing inflammatory skin disease characterized by symptoms including intense pruritus (e.g., severe itch) and by scaly and dry eczematous lesions. Severe disease can be extremely disabling due to major psychological problems, significant sleep loss, and impaired quality of life, leading to high socioeconomic costs. The pathophysiology of AD is influenced by a complex interplay between Immunoglobulin E (IgE)-mediated sensitization, the immune system, and environmental factors. The primary skin defect may be an immunological disturbance that causes IgE-mediated sensitization, with epithelial-barrier dysfunction that is the consequence of both genetic mutations and local inflammation. AD often begins in childhood before age 5 and may persist into adulthood.

Typical treatments for AD include topical lotions and moisturizers, topical corticosteroid ointments, creams or injections. Most treatment options, however, offer only temporary, incomplete, symptom relief. Moreover, many patients with moderate-to-severe AD become resistant to treatment by topical corticosteroids or by calcineurin inhibitors. Thus, a high medical need exists in the art for novel targeted therapies for the treatment and/or prevention of AD.

IL-17C is a secreted homodimer of the IL-17 protein family that was first cloned in 2000 (Li. et al. (2000). Proc. Natl. Acad. Sci. U.S.A. 97, 773-8). It was shown in vitro that IL-17C stimulates the release of TNF-α and IL-1β from the monocytic cell line THP-1 and induces the mRNA expression of inflammatory cytokines such as IL-1β, IL-6 and IL-23 in peritoneal exudate cells (PECs) and the 3T3 cell line (Yamaguchi et al. (2007) J. Immunol 179, 7128-36).

The role of IL-17C as a proinflammatory cytokine relevant for host defense was postulated in several studies (Chang et al. (2011) Immunity 35, 611-621, Song et al. (2011) Nature Immunology 12, 12, Ramirez-Carrozzi et al. (2011) Nature Immunology 12, 12). Also a potential role in the progression of specific tumours and cancerous tissues was recently shown (Song et al. (2014) Immunity 40, 140-152). Only recently, it was demonstrated that the IL 17RE/IL17-RA complex, preferentially expressed in epithelial cells, is the functional receptor for IL-17C and that epithelial cells are the predominant source of IL-17C.

Antibodies that antagonize IL-17C were disclosed (e.g. in WO1999/060127, WO 2013/057241, WO2017/060289, WO2017/140831) and it was demonstrated that antagonists of IL-17C are effective in the treatment of auto-inflammatory disorders, such as rheumatoid arthritis, psoriasis or atopic dermatitis.

MOR106 (WO2017/140831) is a fully human antibody that binds IL-17C and inhibits binding of IL-17C to its receptor throughout relevant species (e.g. human, mouse and cynomolgus monkey) with an IC₅₀ concentration of 80 pM or less. MOR106 proved to be effective in various in vivo mouse models for atopic dermatitis and psoriasis.

The terms MOR106 and MOR00106 are used interchangeably in the present disclosure.

MOR106 was studied in clinical trials of atopic dermatitis. The Phase 1 study was a randomized, double-blind, placebo-controlled trial, evaluating the safety, tolerability and pharmacokinetics of single ascending doses (SAD) in healthy volunteers, and multiple ascending doses (MAD) in patients with moderate-to-severe atopic dermatitis where MOR106 was administered as an intravenous infusion.

The study's secondary objective was to characterize the pharmacokinetic profile of MOR106 in healthy volunteers (SAD part) and patients (MAD part). The second part of the study (MAD part) was not powered for efficacy of MOR106. 24 patients, diagnosed with moderate-to-severe atopic dermatitis, received four weekly infusions of either placebo or MOR106 (1:3). Patients were followed for 11 weeks after the last infusion.

In the SAD part of the Phase 1 study with healthy volunteers as well as in the MAD part with atopic dermatitis patients no clinically relevant safety findings were reported.

In both parts of the Phase 1 study no serious events or infusion related reactions were observed. Adverse events reported were mild to moderate and transient in nature.

The novel antibody demonstrated a dose-dependent PK profile with a half-life in patients in line with what was observed in healthy volunteers. The novel antibody demonstrated a dose-dependent PK profile with a half-life in patients in line with what was observed in healthy volunteers.

Despite the study not being designed to identify efficacy of MOR106 as a primary endpoint, the Inventors have surprisingly identified that in patients with moderate-to-severe atopic dermatitis, who were administered MOR106, 80% (5 of 6) of patients achieved an improvement of the affected area and severity of the skin lesions of more than 50% by week 4 (thus EASI-50 score>80%). Additionally, it was surprisingly found that the effect of MOR106 had a fast onset of action, and that this response was maintained for over 2 months after treatment had stopped.

### SUMMARY

The present disclosure provides a dosing for a pharmaceutical compositions comprising an antibody or antibody fragment specific for IL-17C, in particular MOR106, for use in the treatment of atopic dermatitis (AD), in particular for use in the treatment of subjects suffering from moderate-to-severe atopic dermatitis.

In certain embodiments, the present disclosure provides a pharmaceutical composition comprising a therapeutically effective amount of an antibody or antibody fragment specific for IL-17C for use in the treatment of an AD, wherein said antibody or antibody fragment is administered sequentially at a first dose followed by one or more secondary doses to a subject in need thereof.

In certain embodiments, the subject has moderate-to-severe atopic dermatitis

In certain embodiments, said subject is resistant, non-responsive or inadequately responsive to treatment by either a topical corticosteroid (TCS) or a calcineurin inhibitor.

In certain embodiments, the present disclosure provides a pharmaceutical composition comprising an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15 for use in the treatment of atopic dermatitis, wherein said antibody or antibody fragment is administered at a dose of 0.1 mg/kg-5 mg/kg.

In certain embodiments, the present disclosure provides a pharmaceutical composition for the use disclosed herein, wherein the antibody or antibody fragment is administered at a dose of 3 mg/kg or less.

In certain embodiments, said antibody or antibody fragment is administered intravenously.

In certain embodiments, the present disclosure provides a pharmaceutical composition for the use disclosed herein, wherein said antibody or antibody fragment is administered intravenously at a first dose followed by one or more secondary doses.

In certain embodiments, said antibody or antibody fragment is administered at a first dose of 3 mg/kg or less, wherein the one or more secondary doses each comprise a dose of 3 mg/kg or less.

In certain embodiments, said antibody or antibody fragment is administered once a week, once every two weeks, once every four weeks or once every eight weeks.

In certain embodiments, said antibody or antibody fragment is administered once a week.

In certain embodiments, the present disclosure provides a pharmaceutical composition for the use disclosed herein, wherein said antibody or antibody fragment is administered subcutaneously.

In certain embodiments, said antibody or antibody fragment is administered subcutaneously at a dose of 100 mg-400 mg.

In certain embodiments, said antibody or antibody fragment is administered subcutaneously at a dose of 320 mg or less.

In certain embodiments, said antibody or antibody fragment is administered subcutaneously at a dose of 320 mg.

In certain embodiments, the present disclosure provides a pharmaceutical composition for the use disclosed herein, wherein said use additionally comprises administering said antibody or antibody fragment subcutaneously at a loading dose of 200 mg-800 mg.

In certain embodiments, wherein said antibody or antibody fragment is administered at a loading dose of 640 mg or less.

In certain embodiments, said antibody or antibody fragment is administered at a loading dose of 640 mg or less followed by one or more secondary doses of 320 mg or less.

In certain embodiments, said antibody or antibody fragment is administered subcutaneously once a week, once every two weeks, once every four weeks, or once every eight weeks.

In certain embodiments, the present disclosure provides a pharmaceutical composition for the use disclosed herein, wherein said antibody or antibody fragment is administered at a dose sufficient to achieve a therapeutically effective antibody serum level.

In certain embodiments, wherein said antibody or antibody fragment is administered at a dose sufficient to achieve a serum level of said antibody or antibody fragment of 200 μg/mL or less.

In certain embodiments, said serum level is reached after the first dose administered to a subject in need thereof, said serum level is reached between the first dose and the one or more secondary doses administered to a subject in need thereof.

In certain embodiments, said serum level is maintained during the treatment course.

In certain embodiments, the present disclosure provides a pharmaceutical composition for the use disclosed herein, wherein said use is the treatment of moderate-to-severe atopic dermatitis.

In certain embodiments, said use is the treatment of moderate-to-severe atopic dermatitis of subjects having an EASI score of at least 16 or greater.

In certain embodiments, the present disclosure provides a pharmaceutical composition for the use disclosed herein, wherein the treatment results in an improvement in an atopic dermatitis-associated parameter selected from the group consisting of:
(a) a decrease from baseline in Eczema Area and Severity Index (EASI) score of at least 55%;
(b) a decrease from baseline in SCORAD score of at least 40%
(c) a decrease from baseline in Investigator's Global Assessment (IGA) score of at least 40%.

In certain embodiments, said improvement in an atopic dermatitis-associated parameter is achieved within 12, 8, 4, or 2 weeks after the first administered dose.

In certain embodiments, said improvement in a decrease from baseline in Eczema Area and Severity Index (EASI) score of at least 55% is achieved within 6, 4, or 2 weeks after the first administered dose.

In certain embodiments, said improvement in the decrease from baseline in Eczema Area and Severity Index (EASI) score of at least 55% is maintained for at least 4 weeks after the last administered dose.

In certain embodiments, wherein said improvement in the decrease from baseline in Eczema Area and Severity Index (EASI) score of at least 55% is maintained for at least 4 weeks after the treatment course.

In certain embodiments, the present disclosure provides an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15 for use in the manufacture of a medicament for the treatment of AD, wherein said antibody or antibody fragment is administered at a dose of 0.1 mg/kg-5 mg/kg.

In certain embodiments, said antibody is administered at a dose of 3 mg/kg or less.

In certain embodiments, said antibody is administered intravenously.

In certain embodiments, said antibody is administered subcutaneously.

In certain embodiments, the present disclosure provides a method to treat a patient suffering from atopic dermatitis, said method comprising administering to said patient an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15, wherein said antibody or antibody fragment is administered at a dose of at a dose of 0.1 mg/kg-5 mg/kg.

In certain embodiments, said antibody is administered at a dose of 3 mg/kg or less.

In certain embodiments, said antibody is administered intravenously.

In certain embodiments, said antibody is administered subcutaneously.

In certain embodiments, the present disclosure provides a pharmaceutical composition comprising an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15 for use in the treatment of atopic dermatitis, wherein said antibody or antibody fragment is administered at a dose of 0.1 mg/kg-5 mg/kg.

In certain embodiments the antibody or antibody fragment is administered at a dose of 3 mg/kg or less.

In certain embodiments said antibody or antibody fragment is administered at a first dose followed by one or more secondary doses, and wherein each of said first dose and each of said secondary doses comprise a dose of 3 mg/kg or less.

In certain embodiments, said antibody or antibody fragment is administered subcutaneously at a dose of 100 mg-400 mg.

In certain embodiments, said antibody or antibody fragment is administered subcutaneously at a dose of 320 mg or less.

In certain embodiments, prior to said administration additionally a loading dose of 200 mg-800 mg of said antibody or antibody fragment is administered.

In certain embodiments said loading dose is a loading dose of 640 mg or less.

In certain embodiments said antibody or antibody fragment is administered once a week, once every two weeks, once every four weeks, or once every eight weeks.

In certain embodiments said antibody or antibody fragment is administered at a dose sufficient to achieve a therapeutically effective serum level.

In certain embodiments said serum level is maintained during the treatment course.

In certain embodiments said use is the treatment of moderate-to-severe atopic dermatitis.

In certain embodiments the treatment results in an improvement in an atopic dermatitis-associated parameter selected from the group consisting of:
(a) a decrease from baseline in Eczema Area and Severity Index (EASI) score of at least 55%;
(b) a decrease from baseline in SCORAD score of at least 40%;
(c) a decrease from baseline in Investigator's Global Assessment (IGA) score of at least 40%.

In certain embodiments, said improvement in the decrease from baseline in Eczema Area and Severity Index (EASI) score of at least 55% is maintained for at least 4 weeks after the last administered dose.

In certain embodiments, said improvement in the decrease from baseline in Eczema Area and Severity Index (EASI) score of at least 55% is maintained for at least 4 weeks after the treatment course.

### FIGURE LEGENDS

FIG. 1 shows median percentage EASI score change from baseline in pooled patients (1 mg/kg, 4 mg/kg and 10 mg/kg cohort) administered MOR106 vs. placebo over the course of the study of Example 1.

FIG. 2 shows median percentage change from baseline in EASI patients administered 1 mg/kg or 10 mg/kg of MOR106 in the study of Example 1.

FIG. 3 shows mean percentage SCORAD score change from baseline in patients administered 1 mg/kg or 10 mg/kg of MOR106 for the study of Example 1.

FIG. 4 shows number of patients (pooled patients) achieving IGA=1 for the study of Example 1.

FIG. 5 shows individual patient serum concentrations of MOR106 over the course of the study of Example 1 treated with 1 mg/kg or 10 mg/kg MOR106.

FIG. 6 indicates doses and schedules of a Phase 2 clinical trial assessing efficacy of MOR106 in AD patients administered intravenously or subcutaneously for the study in Example 6.

FIG. 7 indicates simulated MOR106 serum levels of the Phase 2 clinical trial in Example 6.

### DETAILED DESCRIPTION OF THE INVENTION

The term “Atopic Dermatitis” or “AD”, as used herein, means an inflammatory skin disease characterized by intense pruritus (e.g., severe itch) and by scaly and dry eczematous lesions. The term “Atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present disclosure encompasses methods to treat patients with mild, moderate-to-severe or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 20% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art. According to the present disclosure a patient having an EASI score of ≥16 at the screening and baseline visits of the study in Example 1 has been considered to suffer from “moderate-to-severe AD”

In certain embodiment provided herein are methods to treat both the extrinsic and the intrinsic forms of AD. The extrinsic form of AD associated with IgE-mediated sensitization and increased levels of Th2 cytokines involves 70% to 80% of patients with AD. The intrinsic form without IgE-mediated sensitization involves 20% to 30% of patients with AD; these patients have lower levels of IL-4 and IL-13 than extrinsic AD.

The term “IL-17C” refers to a protein known as interleukin 17C (identified in HUGO Gene Nomenclature Committee (HGNC) by ID 5983 and in Mouse genome Informatics (MGI) database by ID 2446486). IL-17C is in some older publications referred to as CX2 or IL-21, however, it should not be confused with IL-21 cytokine, which is specifically expressed in activated CD4⁺ T cells, but not most of other tissues (Parrish-Novak et al (2000). Nature 408 (6808): 57-63). Human IL-21 is located on Chromosome 4 and is identified in HGNC database by ID 6005.

Human IL-17C is located on Chromosome 16 and has the amino acid sequence of (UniProt Q9POM4):

(SEQ ID NO: 1)
MTLLPGLLFLTWLHTCLAHHDPSLRGHPHSHGTPHCYSAEELPLGQAPP
HLLARGAKWGQALPVALVSSLEAASHRGRHERPSATTQCPVLRPEEVLE
ADTHQRSISPWRYRVDTDEDRYPQKLAFAECLCRGCIDARTGRETAALN
SVRLLQSLLVLRRRPCSRDGSGLPTPGAFAFHTEFIHVPVGCTCVLPRS
V

Mouse IL-17C has the amino acid sequence of (UniProt Q8K4C5):

(SEQ ID NO: 4)
MSLLLLGWLPTGMTHQDPPSWGKPRSHRTLRCYSAEELSHGQAPPHLLT
RSARWEQALPVALVASLEATGHRRQHEGPLAGTQCPVLRPEEVLEADTH
ERSISPWRYRIDTDENRYPQKLAVAECLCRGCINAKTGRETAALNSVQL
LQSLLVLRRQPCSRDGTADPTPGSFAFHTEFIRVPVGCTCVLPRSTQ

Cynomolgus monkey IL-17C has the amino acid sequence of (XP_005592825.1):

(SEQ ID NO: 5)
MTLLPGLLFLTWLHACLAHQDPFLRGHPHTHGTPRCYSAEELPLGQAPP
HLLARGAKWGQALPVALVSSLEAAGHRRRHDRPSAATQCPVLRPEEVLE
ADTHQRSISPWRYRVDTDEDRYPQKLAFAECLCRGCIDPRTGRETAALN
SVRLLQSLLVLRRRPCSRDGSGLPTPGAFAFHTEFIRVPVGCTCVLPRS
V

The term “IL-17RA” refers to a protein known as interleukin 17 receptor A. Human IL-17RA has the amino acid sequence of (UniProt Q96F46):

(SEQ ID NO: 2)
MGAARSPPSAVPGPLLGLLLLLLGVLAPGGASLRLLDHRALVCSQPGLN
CTVKNSTCLDDSWIHPRNLTPSSPKDLQIQLHFAHTQQGDLFPVAHIEW
TLQTDASILYLEGAELSVLQLNTNERLCVRFEFLSKLRHHHRRWRFTFS
HFVVDPDQEYEVTVHHLPKPIPDGDPNHQSKNFLVPDCEHARMKVTTPC
MSSGSLWDPNITVETLEAHQLRVSFTLWNESTHYQILLTSFPHMENHSC
FEHMHHIPAPRPEEFHQRSNVTLTLRNLKGCCRHQVQIQPFFSSCLNDC
LRHSATVSCPEMPDTPEPIPDYMPLWWYWFITGISILLVGSVILLIVCM
TWRLAGPGSEKYSDDTKYTDGLPAADLIPPPLKPRKVWIIYSADHPLYV
DVVLKFAQFLLTACGTEVALDLLEEQAISEAGVMTWGRQKQEMVESNSK
IIVLCSRGTRAKWQALLGRGAPVRLRCDHGKPVGDLFTAAMNMILPDFK
RPACFGTYVVCYFSEVSCDGDVPDLFGAAPRYPLMDRFEEVYFRIQDLE
MFQPGRMHRVGELSGDNYLRSPGGRQLRAALDRFRDWQVRCPDWFECEN
LYSADDQDAPSLDEEVFEEPLLPPGTGIVKRAPLVREPGSQACLAIDPL
VGEEGGAAVAKLEPHLQPRGQPAPQPLHTLVLAAEEGALVAAVEPGPLA
DGAAVRLALAGEGEACPLLGSPGAGRNSVLFLPVDPEDSPLGSSTPMAS
PDLLPEDVREHLEGLMLSLFEQSLSCQAQGGCSRPAMVLTDPHTPYEEE
QRQSVQSDQGYISRSSPQPPEGLTEMEEEEEEEQDPGKPALPLSPEDLE
SLRSLQRQLLFRQLQKNSGWDTMGSESEGPSA

The term “IL-17RE” refers to a protein known as interleukin 17 receptor E. Human IL-17RE has the amino acid sequence of (UniProt Q8NFR9):

(SEQ ID NO: 3)
MGSSRLAALLLPLLLIVIDLSDSAGIGFRHLPHWNTRCPLASHTDDSFT
GSSAYIPCRTWWALFSTKPWCVRVWHCSRCLCQHLLSGGSGLQRGLFHL
LVQKSKKSSTFKFYRRHKMPAPAQRKLLPRRHLSEKSHHISIPSPDISH
KGLRSKRTQPSDPETWESLPRLDSQRHGGPEFSFDLLPEARAIRVTISS
GPEVSVRLCHQWALECEELSSPYDVQKIVSGGHTVELPYEFLLPCLCIE
ASYLQEDTVRRKKCPFQSWPEAYGSDFWKSVHFTDYSQHTQMVMALTLR
CPLKLEAALCQRHDWHTLCKDLPNATARESDGWYVLEKVDLHPQLCFKF
SFGNSSHVECPHQTGSLTSWNVSMDTQAQQLILHFSSRMHATFSAAWSL
PGLGQDTLVPPVYTVSQARGSSPVSLDLIIPFLRPGCCVLVWRSDVQFA
WKHLLCPDVSYRHLGLLILALLALLTLLGVVLALTCRRPQSGPGPARPV
LLLHAADSEAQRRLVGALAELLRAALGGGRDVIVDLWEGRHVARVGPLP
WLWAARTRVAREQGTVLLLWSGADLRPVSGPDPRAAPLLALLHAAPRPL
LLLAYFSRLCAKGDIPPPLRALPRYRLLRDLPRLLRALDARPFAEATSW
GRLGARQRRQSRLELCSRLEREAARLADLG

Murine IL17RE has the amino acid sequence of (UniProt Q8BH06):

(SEQ ID NO: 6)
MGSPRLAALLLSLPLLLIGLAVSARVACPCLRSWTSHCLLAYRVDKRFAG
LQWGWFPLLVRKSKSPPKFEDYWRHRTPASFQRKLLGSPSLSEESHRISI
PSSAISHRGQRTKRAQPSAAEGREHLPEAGSQKCGGPEFSFDLLPEVQAV
RVTIPAGPKASVRLCYQWALECEDLSSPFDTQKIVSGGHTVDLPYEFLLP
CMCIEASYLQEDTVRRKKCPFQSWPEAYGSDFWQSIRFTDYSQHNQMVMA
LTLRCPLKLEASLCWRQDPLTPCETLPNATAQESEGWYILENVDLHPQLC
FKFSFENSSHVECPHQSGSLPSWTVSMDTQAQQLTLHFSSRTYATFSAAW
SDPGLGPDTPMPPVYSISQTQGSVPVTLDLIIPFLRQENCILVWRSDVHF
AWKHVLCPDVSHRHLGLLILALLALTALVGVVLVLLGRRLLPGSGRTRPV
LLLHAADSEAQRRLVGALAELLRTALGGGRDVIVDLWEGTHVARIGPLPW
LWAARERVAREQGTVLLLWNCAGPSTACSGDPQAASLRTLLCAAPRPLLL
AYFSRLCAKGDIPRPLRALPRYRLLRDLPRLLRALDAQPATLASSWSHLG
AKRCLKNRLEQCHLLELEAAKDDYQGSTNSPCGFSCL

The term “antibody” as used herein refers to a protein comprising at least two heavy (H) chains and two light (L) chains inter-connected by disulfide bonds which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

A “human antibody” or “human antibody fragment”, as used herein, includes antibodies and antibody fragments having variable regions in which both the framework and CDR regions are derived from sequences of human origin. Human antibodies can also be isolated from synthetic libraries or from transgenic mice (e.g. xenomouse) provided the respective system yield in antibodies having variable regions in which both the framework and CDR regions are equivalent to the sequences of human origin.

Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86).

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and AI-Lazikani et al., (1997) J. Mol. Biol. 273:927-948.

A “humanized antibody” or “humanized antibody fragment” is defined herein as an antibody molecule which has constant antibody regions derived from sequences of human origin and the variable antibody regions or parts thereof or only the CDRs are derived from another species. For example a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated” refers to a compound, which can be e.g. an antibody or antibody fragment, that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody”, as used herein, includes all antibodies that are prepared, expressed, created or segregated by means not existing in nature. For example antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germLine immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germLine VH and VL sequences, may not naturally exist within the human antibody germLine repertoire in vivo. A recombinant antibody may be a monoclonal antibody. In an embodiment, the antibodies and antibody fragment disclosed herein are isolated from the Ylanthia® antibody library as disclosed in U.S. Ser. No. 13/321,564 or U.S. Ser. No. 13/299,367, which both herein are incorporated by reference.

The term “monoclonal antibody” as used herein refers to a preparation of antibody molecules of single molecular composition. A monoclonal antibody composition displays a unique binding site having a unique binding specificity and affinity for particular epitopes.

The terms “antagonist of IL-17C” and an “IL-17C antagonist”, are used interchangeably herein and refer to any molecule which inhibits the activity or function of IL 17C. The term “IL 17C antagonist” includes, but is not limited to, antibodies or antibody fragments specifically binding to IL-17C. Preferably, an IL-17C antagonist in the present disclosure is an antibody specific for human IL-17C. Such an antibody may be of any type, such as a murine, a rat, a chimeric, a humanized or a human antibody.

The term “antagonistic antibody specific for IL-17C” or “antagonistic antibodies specific for IL-17C” refers to antibodies or antibody fragments specifically binding to IL-17C.

More preferably an IL-17C antagonist is an antibody or antibody fragment, such as a monoclonal antibody, specifically binding to IL-17C and blocks the binding of IL-17C to receptors of IL-17C, wherein the receptors of IL-17C include IL-17RE and IL-17RA. Such an antibody may be of any type, such as a murine, a rat, a chimeric, a humanized or a human antibody.

As used herein, an antibody “binds specifically to”, “specifically binds to”, is “specific to/for” or “specifically recognizes” an antigen or epitope if such antibody is able to discriminate between such antigen or epitope and one or more reference antigen(s) or epitope(s), since binding specificity is not an absolute, but a relative property. For example, a standard ELISA assay can be carried out. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

Compositions of the present disclosure may be used for therapeutic or prophylactic applications. The present disclosure, therefore, includes a pharmaceutical composition containing an antibody (or functional antibody fragment) as disclosed herein and a pharmaceutically acceptable carrier or excipient therefor. In a related aspect, the present disclosure provides a method for treating atopic dermatitis, e.g., moderate to severe atopic dermatitis Such method contains the steps of administering to a subject in need thereof an effective amount of the pharmaceutical composition that contains an antibody (or functional antibody fragment) as described or contemplated herein.

The present disclosure provides therapeutic methods comprising the administration of a therapeutically effective amount of an IL-17C antibody as disclosed to a subject in need of such treatment. A “therapeutically effective amount” or “effective amount”, as used herein, refers to the amount of an antibody specific for IL-17C, necessary to elicit the desired biological response. In accordance with the subject disclosure, the therapeutic effective amount is the amount of an antibody specific for IL-17C necessary to treat and/or prevent atopic dermatitis and symptoms associated with atopic dermatitis.

As used herein, the terms “treat”, “treating”, or the like, mean to alleviate symptoms, eliminate the causation of symptoms either on a temporary or permanent basis, or to prevent or slow the appearance of symptoms of the named disorder or condition.

As used herein, the terms “subject”, “a subject in need thereof” or the like, mean a human or non-human animal that exhibits one or more symptoms or indicia of atopic dermatitis, and/or who has been diagnosed with atopic dermatitis. Preferably the subject is a patient who has been diagnosed with atopic dermatitis. In certain embodiments, the methods may be used to treat patients that show elevated levels of one or more AD-associated biomarkers (described elsewhere herein). For example, the methods provided herein comprise administering an IL-17C antagonist to patients with elevated levels of IgE or TARC or periostin. “Subject” or “species”, as used in this context refers to any mammal, including rodents, such as mouse or rat, and primates, such as cynomolgus monkey (Macaca fascicularis), rhesus monkey (Macaca mulatta) or humans (Homo sapiens). Preferably the subject is a primate, most preferably a human.

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Pharmacokinetics” or “PK” as used herein describes how the body affects a specific drug after administration through the mechanisms like absorption and distribution, as well as the metabolic changes of the drug in the body, and the effects and routes of excretion of the metabolites of the drug. Pharmacokinetic properties of drugs may be affected by the route of administration and the dose of administered drug.

### Further Embodiments

In certain embodiments, the present disclosure provides a pharmaceutical composition comprising a therapeutically effective amount of antibody or antibody fragment specific for IL17-C for use in the treatment of atopic dermatitis, wherein said antibody or antibody fragment is administered at a first dose followed by one or more secondary doses to a subject in need thereof.

In certain embodiments, said subject has moderate-to-severe atopic dermatitis.

In certain embodiments, said antibody or antibody fragment is administered sequentially at a first dose followed by one or more secondary doses to a subject in need thereof.

### Dosage

The dose of an antibody or antibody fragment comprised in a pharmaceutical composition according to the present disclosure administered to a patient may vary depending upon the age and the size of the patient, symptoms, conditions, route of administration, and the like. The dose is typically calculated according to body weight, or body surface area, age, or per individual. Depending on the severity of the condition, the frequency and the duration of the treatment can be adjusted. Effective dosages and schedules for administering pharmaceutical compositions comprising antibodies or antibody fragments specific for IL-17C may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The pharmaceutical composition may include dosage forms for intravenous, subcutaneous, intracutaneous and intramuscular injections, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Exemplary pharmaceutical compositions comprising an antibody or antibody fragment specific for IL-17C that can be used in the context of the present invention are disclosed, e.g., in WO2017140831.

In certain ways of administration, e.g. intravenous administration, it is preferred to administer drug depending on the body weight of the patient. In other ways of administration, e.g. subcutaneous administration, it is preferred to administer drug at a flat, fixed does. The skilled person is aware of which dose in one way of administration is equivalent to another dose in another way of administration. The pharmacodynamics of a specific drug are typically taken into account in a reasoned decision to administer a drug in the required from and at a required, efficacious dose.

### Amount and Doses

The amount of an antibody or antibody fragment specific for IL-17C administered to a subject according to the present disclosure is, generally, a therapeutically effective amount.

The amount of an antibody or antibody fragment specific for IL-17C according to the present disclosure contained within the individual doses for a subcutaneous application is usually expressed in terms of milligrams (i.e., mg).

A therapeutically effective amount of an antibody or antibody fragment specific for IL-17C according to the present disclosure contained within the individual doses of a subcutaneous application can be 100-400 mg.

A therapeutically effective amount of an antibody or antibody fragment specific for IL-17C according to the present disclosure contained within the individual doses of a subcutaneous application can be 320 mg or less.

A therapeutically effective amount of an antibody or antibody fragment specific for IL-17C according to the present disclosure contained within the individual doses of a subcutaneous application can be 240 mg or less.

A therapeutically effective amount of an antibody or antibody fragment specific for IL-17C according to the present disclosure contained within the individual doses of a subcutaneous application can be 160 mg or less.

In certain embodiments, a dose of 320 mg or less of an antibody or antibody fragment specific for IL-17C is administered to a subject.

In certain embodiments, said individual dose is administered subcutaneously.

In certain embodiments, said individual dose is administered subcutaneously as a flat dose e.g. as a single injection.

In certain embodiments, said individual dose is administered subcutaneously as a flat dose comprised in a formulation volume of 2 ml.

In certain embodiments, said antibody or antibody fragment is additionally administered subcutaneously with a loading dose.

In certain embodiments, said antibody or antibody fragment is administered subcutaneously with a loading dose followed by one or more secondary doses.

In certain embodiments, said antibody or antibody fragment is administered subcutaneously with a loading dose of 200-800 mg followed by one or more secondary doses of 100-400 mg.

In certain embodiments, said antibody or antibody fragment is administered subcutaneously with a loading dose of 640 mg or less followed by one or more secondary doses of 320 mg or less.

From other therapeutic antibodies it is known that the same amount of an antibody when administered intravenously corresponds to an average of approximately 60% (i.e., 50-76%) of the exposure (i.e. area under the time vs. drug concentration curve) of the antibody in the serum when the same antibody amount is administered subcutaneously (Meibohm, B.: Pharmacokinetics and Pharmacodynamics of Biotech Drugs, Wiley-VCH, 2006). Therefore, the concentration of a subcutaneous formulation needs to be higher to achieve the same drug serum level or exposure as compared to an intravenous formulation.

As such, in certain embodiments, the therapeutically effective amount of an antibody or antibody fragment contained within the individual doses of a subcutaneously administered antibody is equivalent to the therapeutically effective amount of said antibody or antibody fragment in the individual doses administered intravenously to a subject in need thereof.

In the case of a pharmaceutical composition comprising an antibody or antibody fragment specific for IL-17C according to the present disclosure, a therapeutically effective amount of said antibody or antibody fragment contained within the individual doses for an intravenous application may be also expressed in terms of milligrams of antibody per kilogram of patient body weight (i.e., mg/kg).

In certain embodiments, the therapeutically effective amount of an antibody or antibody fragment contained within the individual doses of a subcutaneously administered antibody is equivalent to the therapeutically effective amount of said antibody or antibody fragment in the individual doses administered intravenously to a subject in need thereof.

In certain embodiments, said individual dose administered is a dose of 5 mg/kg-0.1 mg/kg

In certain embodiments, said individual dose administered is a dose of 5 mg/kg-1 mg/kg, 3 mg/kg-1 mg/kg, 2 mg/kg-1 mg/kg, or 1 mg/kg or less.

In certain embodiments, said individual dose administered is a dose of 5 mg/kg-1 mg/kg, 4 mg/kg-2 mg/kg, or 3 mg/kg.

In certain embodiments, a dose of 5 mg/kg or less, 4 mg/kg or less, 3 mg/kg or less, 1 mg/kg or less of an antibody or antibody fragment specific for IL-17C is administered to a subject.

In certain embodiments, a dose of 5 mg/kg or less of an antibody or antibody fragment specific for IL-17C is administered to a subject.

In certain embodiments, a dose of 4 mg/kg or less of an antibody or antibody fragment specific for IL-17C is administered to a subject.

In certain embodiments, a dose of 3 mg/kg or less of an antibody or antibody fragment specific for IL-17C is administered to a subject.

In certain embodiments, a dose of 2 mg/kg or less of an antibody or antibody fragment specific for IL-17C is administered to a subject.

In certain embodiments, a dose of 1 mg/kg or less of an antibody or antibody fragment specific for IL-17C is administered to a subject.

In certain embodiments, said antibody or antibody fragment is administered intravenously.

In certain embodiments, said antibody or antibody fragment is administered subcutaneously.

### Serum Levels

As indicated above, it is estimated that an amount of MOR106 that leads to a certain level of said antibody in the serum when administered intravenously corresponds to approximately 60% of the serum exposure achieved when the amount of MOR106 is administered subcutaneously. Therefore, the drug amount of a subcutaneous injection needs to be higher to achieve the same drug serum level or exposure as compared to an intravenous formulation.

As such, in certain embodiments the serum level to be achieved in a subject is equal or higher compared to the serum concentration achieved with intravenous administration of the antibody or antibody fragment of the present disclosure at a dose of 0.1 mg/kg-5 mg/kg when administered once a week over at least four weeks.

As such, in certain embodiments the serum level to be achieved in a subject is equal or higher compared to the serum concentration achieved with intravenous administration of the antibody or antibody fragment of the present disclosure at a dose of 5 mg/kg or less, of 4 mg/kg or less, 3 mg/kg or less, 2 mg/kg or less, or of 1 mg/kg or less when administered once a week over at least four weeks.

In certain embodiments, the serum level to be achieved in a subject is equal or higher compared to the serum concentration achieved with intravenous administration of the antibody or antibody fragment of the present disclosure at a dose of 5 mg/kg-1 mg/kg, 3 mg/kg-1 mg/kg, 2 mg/kg-1 mg/kg, or 1 mg/kg or less when administered once a week over at least four weeks..

In certain embodiments, the serum level to be achieved in a subject is equal or higher compared to the serum concentration achieved with intravenous administration of the antibody or antibody fragment of the present disclosure at a dose of 5 mg/kg-1 mg/kg, 4 mg/kg-2 mg/kg, or 3 mg/kg when administered once a week over at least four weeks.

In certain embodiments, the serum level to be achieved in a subject is equal or higher compared to the serum concentration achieved with intravenous administration of the antibody or antibody fragment of the present disclosure at a dose of 5 mg/kg or less, when administered once a week over at least four weeks.

In certain embodiments, the serum level to be achieved in a subject is equal or higher compared to the serum concentration achieved with intravenous administration of the antibody or antibody fragment of the present disclosure at a dose of 3 mg/kg or less when administered once a week over at least four weeks.

In certain embodiments, the present disclosure provides an pharmaceutical composition comprising an antibody or antibody fragment specific for IL-17C for use in the treatment of AD, wherein said antibody or antibody fragment is administered at a dose to achieve a therapeutically effective antibody level in the serum equal or higher compared to the intravenous administration of said antibody at a dose of 5 mg/kg or less, of 4 mg/kg or less, of 3 mg/kg or less, or 1 mg/kg or less.

In certain embodiments, said antibody or antibody fragment is administered once in a week, once in 2 weeks, once in 3 weeks, once in 4 weeks, once in 8 weeks, or once in 16 weeks.

In certain embodiments, said antibody or antibody fragment is administered once a week.

In certain embodiments, said antibody or antibody fragment is administered once a week for at least four weeks.

In certain embodiments, the dose of an antibody or antibody fragment specific for IL-17C administered to a subject is sufficient to achieve a serum level of said antibody or antibody fragment of 5000 ng/mL or more during the treatment course.

In certain embodiments, the dose of an antibody or antibody fragment specific for IL-17C administered to a subject is sufficient to achieve a serum level of said antibody or antibody fragment of 200 μg/mL or less In certain embodiments, the dose of an antibody or antibody fragment specific for IL-17C administered to a subject is sufficient to achieve a serum level of said antibody or antibody fragment of 500 μg/mL or less, 400 μg/mL or less, 300 μg/mL or less, 200 μg/mL, or 100 μg/mL or less.

In certain embodiments, the dose of an antibody or antibody fragment specific for IL-17C administered to a subject is sufficient to maintain a serum level of said antibody or antibody fragment of 200 μg/mL or less over the treatment course.

In an embodiment, the dose of said antibody or antibody fragment and the dosing frequency is sufficient to achieve and maintain a serum concentration of said antibody or antibody fragment of 200 μg/ml or less over the treatment course.

In certain antibody, said antibody or antibody fragment is administered intravenously at a dose of 3 mg/kg once a week.

In certain antibody, said antibody or antibody fragment is administered subcutaneously at a dose of 320 mg or less once weekly.

In certain antibody, said antibody or antibody fragment is additionally administered subcutaneously with a loading dose of 200-800 mg followed by a dose of 100-400 mg once a week.

In certain antibody, said antibody or antibody fragment is additionally administered subcutaneously with a loading dose of 640 mg or less followed by a dose of 320 mg or less once a week.

In certain antibody, said antibody or antibody fragment is administered subcutaneously with a loading dose of 640 mg or less followed by a dose of 320 mg or less once every 2 weeks.

In certain antibody, said antibody or antibody fragment is administered subcutaneously with a loading dose of 640 mg or less followed by a dose of 320 mg or less once every 4 weeks.

In certain antibody, said antibody or antibody fragment is administered subcutaneously with a loading dose of 640 mg or less followed by a dose of 320 mg or less once every 8 weeks.

In certain antibody, said antibody or antibody fragment is administered subcutaneously with a loading dose of 640 mg or less followed by a dose of 320 mg or less once every 16 weeks.

In certain embodiments, said antibody or antibody fragment is administered subcutaneously at a loading dose of 640 mg or less followed by one or more secondary doses of 320 mg or less.

In certain embodiments, the dose of an antibody or antibody fragment specific for IL-17C administered to a subject is sufficient to achieve a serum level of said antibody or antibody fragment of 100 ng/mL or more.

In certain embodiments, said serum level of said antibody or antibody fragment specific for IL-17C is achieved after the treatment course.

In certain embodiments, said serum level of said antibody or antibody fragment specific for IL-17C is achieved at day 99 after the first dose.

In certain embodiments, said dose is administered intravenously or subcutaneously.

### Dosing Frequency

In certain embodiments, therapeutically effective amount of the antibody or antibody fragment specific for IL-17C is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In certain embodiments, the therapeutically effective amount of the antibody or antibody fragment specific for IL-17C is administered to a subject at a dosing frequency of about once a week.

In certain embodiments, the therapeutically effective amount of the antibody or antibody fragment specific for IL-17C is administered once a week, for at least four weeks.

In certain embodiments, the therapeutically effective amount of the antibody or antibody fragment specific for IL-17C is administered once a week, once every 2 weeks, once every 3 weeks, once every 4 weeks, once every 8 weeks, or once every 16 weeks According to certain embodiments of the present disclosure, multiple doses of a therapeutically effective amount of the antibody or antibody fragment specific for IL-17C may be administered to a subject over a defined time course.

According to certain embodiments of the present disclosure, the antibody or antibody fragment specific for IL-17C may be sequentially administered to a subject over a defined time course.

As used herein, “sequentially administered” means that each dose of the antibody or antibody fragment specific for IL-17C is administered to the subject at a different point in time, e.g., on different days separated by a predetermined interval (e.g., hours, days, weeks or months).

In certain embodiments, the antibody or antibody fragment specific for IL-17C is sequentially administered to a subject at a single first dose of an antibody or antibody fragment specific for IL-17C, followed by one or more secondary doses of said antibody or antibody fragment.

The terms “first dose,” and “secondary doses,” refer to the temporal sequence of administration of the antibody specific for IL-17C. Thus, the “first dose” is the dose which is administered at the beginning of the treatment regimen (also referred to as the “baseline dose”); the “secondary doses” are the doses which are administered after the first dose; The first and secondary doses may all contain the same amount of antibody or antibody fragment specific for IL-17C or may differ from one another in terms of frequency of administration.

In certain embodiments, the amount of antibody or antibody fragment specific for IL-17C contained in the first and secondary doses varies from one another (e.g., adjusted up or down as appropriate) during the course of treatment.

In other embodiments, the amount of antibody or antibody fragment specific for IL-17C contained in the first and secondary doses are invariant from one another (e.g., maintaining the amount) during the course of treatment.

In certain embodiments, the first dose comprises a first amount of the antibody or antibody fragment and the one or more secondary doses each comprise a second amount of the antibody or antibody fragment.

In certain embodiments, the first amount of antibody or antibody fragment is 1×, 1.5×, 2×, 2.5×, 3×, 3.5×, 4×, or 5×the second amount of the antibody or antibody fragment.

In certain embodiments, wherein the antibody or antibody fragment is administered subcutaneously, one or more (e.g., 1, 2, 3, 4, or 5) doses may be administered at the beginning of the treatment regimen as loading doses followed by subsequent doses that are administered on a less frequent basis.

A “loading dose” as used herein is a bolus (mg/kg, mg, etc.) of drug that to achieve a therapeutically effective serum level of the antibody or antibody fragment. A “loading dose” is usually followed one or more secondary doses to maintain the desired effect.

In certain embodiments, two doses are administered at the beginning of the treatment as loading doses followed by one or more secondary doses.

For example, an antibody or antibody fragment specific for IL-17C may be administered to a subject in need thereof at a loading dose of 640 mg or less followed by one or more secondary doses of 320 mg or less.

In certain embodiments, each dose administered at the beginning of the treatment as loading doses comprises a dose of 640 mg.

In certain embodiments, said antibody or antibody fragment specific for IL-17C may be administered to a subject in need thereof at a loading dose of 640 mg or less.

In certain embodiments, said antibody or antibody fragment specific for IL-17C may be administered to a subject in need thereof at a loading dose of 640 mg or less followed by one or more secondary doses of 320 mg or less.

In certain embodiments, said antibody or antibody fragment specific for IL-17C may be administered to a subject in need thereof at a loading dose of 640 mg followed by one or more secondary doses of 320 mg.

In embodiments, the first dose is administered at a dose of 1 mg/kg or less, wherein the one or more secondary doses each comprise of 1 mg/kg or less of the antibody or antibody fragment specific for IL-17C.

In certain embodiments, said antibody or antibody fragment specific for IL-17C is administered at a first dose of 3 mg/kg or less followed by one or more secondary doses wherein each secondary dose comprises of 3 mg/kg or less.

In certain embodiment, each secondary dose is administered 1 week after the immediately preceding dose.

In certain embodiment, each secondary dose is administered 2 weeks after the immediately preceding dose.

In certain embodiment, each secondary dose is administered 4 weeks after the immediately preceding dose.

In certain embodiment, each secondary dose is administered 8 weeks after the immediately preceding dose.

In certain embodiments, the pharmaceutical composition according to the present disclosure comprises said antibody or antibody fragment specific for IL-17C, wherein the antibody or antibody fragment is administered at a dosing frequency of, e.g., once a week, once every 2 weeks, once every 3 weeks, once every 4 weeks, once every 8 weeks, or once every 16 weeks.

In certain embodiment said secondary doses is administered once a week.

The phrase “the immediately preceding dose,” as used herein, means, in a sequence of multiple administrations, the dose of the antibody or antibody fragment specific for IL-17C which is administered to a patient prior to the administration of the very next dose in the sequence with no intervening doses.

In an embodiment, any number of secondary doses of an antibody or antibody fragment specific for IL-17C may be administered to a subject in need thereof, For example, in certain embodiments, only a single secondary dose is administered to the subject.

In certain embodiments, two or more (e.g., 2, 3, 4, 5, 6, 7, 8, or more) secondary doses are administered to the subject.

In embodiments involving multiple secondary doses, each secondary dose may be administered at the same frequency as the other secondary doses. For example, each secondary dose may be administered to the patient 1 to 8 weeks after the immediately preceding dose.

Alternatively, the frequency at which the secondary doses are administered to a patient can vary over the course of the treatment.

In certain embodiments, said dose or doses is/are administered intravenously or subcutaneously.

In certain embodiments, said dose or doses is/are administered subcutaneously.

In certain embodiments, said dose or doses are fixed doses which may be administered once a week, once every 2 weeks, once every 3 weeks, once every 4 weeks, once every 8 weeks, or once every 16 weeks.

### AD-Associated Parameters

According to certain embodiments, the present disclosure provides a pharmaceutical composition comprising a therapeutically effective amount of an antibody or antibody fragment specific for IL-17C for use in the treatment of AD, wherein the treatment results in an improvement in an AD-associated parameter

In certain embodiments, the improvements in AD-associated parameters include, e.g.:
(i) a decrease in Eczema Area and Severity Index (EASI) score;
(ii) a decrease in SCORAD score;
(iii) a decrease in Investigator's Global Assessment (IGA) score;

In certain embodiments, the improvement in an AD-associated parameter is selected from the group consisting of:
(i) a decrease from baseline in EASI score of at least 45%;
(ii) a decrease from baseline in SCORAD score of at least 30%;
(iii) a decrease from baseline in IGA score of at least 25%;
(iv) percent responders with ≥50% improvement in EASI (EASI50).

In certain embodiments, the improvement in an AD-associated parameter is selected from the group consisting of:
(i) a decrease from baseline in EASI score of at least 45%;
(ii) a decrease from baseline in IGA score of at least 25%;
(iii) a decrease from baseline in SCORAD score of at least 30%;
(iv) percent responders with ≥50% improvement in EASI (EASI50).

According to certain embodiments, the present disclosure provides a pharmaceutical composition comprising a therapeutically effective amount of an antibody or antibody fragment specific for IL-17C for use in the treatment of AD, wherein the treatment results in an improvement in an AD-associated parameter, wherein the improvement in the AD-associated parameter is selected from the group consisting of:
(a) a decrease from baseline in Eczema Area and Severity Index (EASI) score of at least 55%;
(b) a decrease from baseline in SCORAD score of at least 40%;
(c) a decrease from baseline in Investigator's Global Assessment (IGA) score of at least 40%;

In certain embodiments, said antibody or antibody fragment is administered intravenously at a dose of 3 mg/kg or less.

In certain embodiments, said antibody or antibody fragment is administered subcutaneously at a dose of 100-400 mg.

In certain embodiments, said antibody or antibody fragment is administered subcutaneously at a dose of 320 mg or less.

In certain embodiments, said antibody or antibody fragment is administered subcutaneously at a dose of 260 mg or less.

In certain embodiments, said antibody or antibody fragment is administered subcutaneously at a dose of 140 mg or less.

In certain embodiments, said antibody or antibody fragment is administered once a week.

In certain embodiments, said antibody or antibody fragment is administered subcutaneously at a dose equivalent to the therapeutically effective amount of said antibody or antibody fragment administered intravenously.

In certain embodiments, said antibody or antibody fragment is additionally administered subcutaneously at a loading dose of 200-800 mg followed by one or more secondary doses of 100-400 mg.

In certain embodiments, said antibody or antibody fragment is additionally administered subcutaneously at a loading dose of 640 mg or less followed by one or more secondary doses of 320 mg or less.

According to certain embodiments, the present disclosure provides a method for improving one or more AD-associated parameter(s) in a subject in need thereof.

### Antibody

In certain embodiment of the present disclosure, the antibody or antibody fragment specific for IL-17C according to the present disclosure comprises a variable heavy chain variable region, a variable light chain region, heavy chain, light chain and/or CDRs comprising any of the amino acid sequences of the IL-17C specific antibodies as set forth in WO2017/140831.

In an embodiment, said antibody or antibody fragment specific for IL-17C comprises a HCDR1 region comprising the amino acid sequence of SEQ ID NO: 7, a HCDR2 region comprising the amino acid sequence of SEQ ID NO: 8, a HCDR3 region comprising the amino acid sequence of SEQ ID NO: 9, a LCDR1 region comprising the amino acid sequence of SEQ ID NO: 13, a LCDR2 region comprising the amino acid sequence of SEQ ID NO: 14 and a LCDR3 region comprising the amino acid sequence of SEQ ID NO: 15.

In one embodiment, said antibody or antibody fragment specific for IL-17C, comprises the HCDR1 region of SEQ ID NO: 7, the HCDR2 region of SEQ ID NO: 8, the HCDR3 region of SEQ ID NO: 9, the LCDR1 region of SEQ ID NO: 13, the LCDR2 region of SEQ ID NO: 14 and the LCDR3 region of SEQ ID NO: 15.

In an embodiment, said antibody or antibody fragment specific for IL-17C, comprises a HCDR1 region comprising the amino acid sequence of SEQ ID NO: 10, a HCDR2 region comprising the amino acid sequence of SEQ ID NO: 11, a HCDR3 region comprising the amino acid sequence of SEQ ID NO: 12, a LCDR1 region comprising the amino acid sequence of SEQ ID NO: 13, a LCDR2 region comprising the amino acid sequence of SEQ ID NO: 14 and a LCDR3 region comprising the amino acid sequence of SEQ ID NO: 15.

In an embodiment, said antibody or antibody fragment specific for IL-17C, comprises the HCDR1 region of SEQ ID NO: 10, the HCDR2 region of SEQ ID NO: 11, the HCDR3 region of SEQ ID NO: 12, the LCDR1 region of SEQ ID NO: 13, the LCDR2 region of SEQ ID NO: 14 and the LCDR3 region of SEQ ID NO: 15.

In an embodiment, said antibody or antibody fragment specific for IL-17C, comprises
(a) a HCDR1 region comprising the amino acid sequence of SEQ ID NO: 7, a HCDR2 region comprising the amino acid sequence of SEQ ID NO: 8, a HCDR3 region comprising the amino acid sequence of SEQ ID NO: 9, a LCDR1 region comprising the amino acid sequence of SEQ ID NO: 13, a LCDR2 region comprising the amino acid sequence of SEQ ID NO: 14 and a LCDR3 region comprising the amino acid sequence of SEQ ID NO: 15, or
(b) a HCDR1 region comprising the amino acid sequence of SEQ ID NO: 10, a HCDR2 region comprising the amino acid sequence of SEQ ID NO: 11, a HCDR3 region comprising the amino acid sequence of SEQ ID NO: 12, a LCDR1 region comprising the amino acid sequence of SEQ ID NO: 13, a LCDR2 region comprising the amino acid sequence of SEQ ID NO: 14 and a LCDR3 region comprising the amino acid sequence of SEQ ID NO: 15.

In an embodiment, said antibody or antibody fragment specific for IL-17C, comprises
(a) the HCDR1 region of SEQ ID NO: 7, the HCDR2 region of SEQ ID NO: 8, the HCDR3 region of SEQ ID NO: 9, the LCDR1 region of SEQ ID NO: 13, the LCDR2 region of SEQ ID NO: 14 and the LCDR3 region of SEQ ID NO: 15, or
(b) the HCDR1 region of SEQ ID NO: 10, the HCDR2 region of SEQ ID NO: 11, the HCDR3 region of SEQ ID NO: 12, the LCDR1 region of SEQ ID NO: 13, the LCDR2 region of SEQ ID NO: 14 and the LCDR3 region of SEQ ID NO: 15.

In an embodiment, said antibody or antibody fragment specific for IL-17C, comprises
(a) the HCDR1 region of SEQ ID NO: 7, the HCDR2 region of SEQ ID NO: 8, the HCDR3 region of SEQ ID NO: 9, the LCDR1 region of SEQ ID NO: 13, the LCDR2 region of SEQ ID NO: 14 and the LCDR3 region of SEQ ID NO: 15, and further comprises a heavy chain of SEQ ID NO: 17 or a light chain of SEQ ID NO: 16, or
(b) the HCDR1 region of SEQ ID NO: 10, the HCDR2 region of SEQ ID NO: 11, the HCDR3 region of SEQ ID NO: 12, the LCDR1 region of SEQ ID NO: 13, the LCDR2 region of SEQ ID NO: 14 and the LCDR3 region of SEQ ID NO: 15 and further comprises a variable heavy chain of SEQ ID NO: 17 or a variable light chain of SEQ ID NO: 16.

In an embodiment, said antibody or antibody fragment specific for IL-17C comprises a variable heavy chain of SEQ ID NO: 17 and a variable light chain of SEQ ID NO: 16.

In a further embodiment, said antibody or antibody fragment specific for IL-17C, comprises a heavy chain of SEQ ID NO: 43 and a light chain of SEQ ID NO: 42.

In one embodiment, the present disclosure refers to a nucleic acid composition comprising a nucleic acid sequence or a plurality of nucleic acid sequences encoding said antibody or antibody fragment specific for IL-17C, wherein said antibody or antibody fragment comprises
(a) the HCDR1 region of SEQ ID NO: 7, the HCDR2 region of SEQ ID NO: 8, the HCDR3 region of SEQ ID NO: 9, the LCDR1 region of SEQ ID NO: 13, the LCDR2 region of SEQ ID NO: 14 and the LCDR3 region of SEQ ID NO: 15, or
(b) the HCDR1 region of SEQ ID NO: 10, the HCDR2 region of SEQ ID NO: 11, the HCDR3 region of SEQ ID NO: 12, the LCDR1 region of SEQ ID NO: 13, the LCDR2 region of SEQ ID NO: 14 and the LCDR3 region of SEQ ID NO: 15,

In another embodiment said antibody or antibody fragment comprises a variable heavy chain of SEQ ID NO: 17 and a variable light chain of SEQ ID NO: 16 or a variable heavy chain and a variable light chain that has at least 60%, at least 70%, at least 80%, at least 90% or at least 95% identity to the a variable heavy chain of SEQ ID NO: 17 and to the variable light chain of SEQ ID NO: 16.

In another embodiment, the disclosure refers to an isolated nucleic acid encoding a heavy chain sequence and/or light chain sequence of said antibody or antibody fragment that binds to IL-17C, the nucleic acid comprising
(a) a HCDR1 region of SEQ ID NO: 58, the HCDR2 region of SEQ ID NO: 59, the HCDR3 region of SEQ ID NO: 60, the LCDR1 region of SEQ ID NO: 64, the LCDR2 region of SEQ ID NO: 65 and the LCDR3 region of SEQ ID NO: 66, or
(b) a HCDR1 region of SEQ ID NO: 61, the HCDR2 region of SEQ ID NO: 62, the HCDR3 region of SEQ ID NO: 63, the LCDR1 region of SEQ ID NO: 64, the LCDR2 region of SEQ ID NO: 65 and the LCDR3 region of SEQ ID NO: 66, or
(c) a HCDR1 region of SEQ ID NO: 33, the HCDR2 region of SEQ ID NO: 34, the HCDR3 region of SEQ ID NO: 35, the LCDR1 region of SEQ ID NO: 39, the LCDR2 region of SEQ ID NO: 40 and the LCDR3 region of SEQ ID NO: 41, or
(d) a HCDR1 region of SEQ ID NO: 36, the HCDR2 region of SEQ ID No: 37, the HCDR3 region of SEQ ID No: 38, the LCDR1 region of SEQ ID No: 39, the LCDR2 region of SEQ ID No: 40 and the LCDR3 region of SEQ ID No: 41.

In an embodiment, the disclosure refers to a nucleic acid encoding an isolated monoclonal antibody or antibody fragment specific for IL-17C, wherein the nucleic acid comprises a VH region of SEQ ID NO: 19 and a VL region of SEQ ID NO: 18, or a VH region and a VL region that has at least 60%, at least 70%, at least 80%, at least 90% or at least 95% identity to the VH region of SEQ ID NO: 19 and/or the VL region of SEQ ID NO: 18.

In an embodiment, the disclosure refers to a nucleic acid encoding an isolated monoclonal antibody or antibody fragment specific for IL-17C, wherein the nucleic acid comprises a VH region of SEQ ID NO: 68 and a VL region of SEQ ID NO: 67, or a VH region and a VL region that has at least 60%, at least 70%, at least 80%, at least 90% or at least 95% identity to the VH region of SEQ ID NO: 68 and/or the VL region of SEQ ID NO: 67.

In an embodiment, the disclosure refers to a nucleic acid encoding an isolated monoclonal antibody or antibody fragment specific for IL-17C wherein the nucleic acid comprises a Heavy chain (IgG1) of SEQ ID NO: 45 and a Light chain of SEQ ID NO: 44.

In an embodiment, the disclosure refers to a nucleic acid encoding an isolated monoclonal antibody or antibody fragment specific for IL-17C wherein the nucleic acid comprises a Heavy chain (IgG1) of SEQ ID NO: 70 and a Light chain of SEQ ID NO: 69.

In an embodiment, the disclosure refers to a nucleic acid encoding an isolated monoclonal antibody or antibody fragment specific for IL-17C wherein the nucleic acid comprises a VH and a VL of any of the antibodies in Table 1.

In an embodiment, the disclosure refers to a nucleic acid encoding an isolated monoclonal antibody or antibody fragment specific for IL-17C wherein the nucleic acid comprises a Heavy chain (IgG1) and a Light chain of any of the antibodies in Table 1.

In an embodiment, the present disclosure refers to a pharmaceutical composition comprising an antibody or antibody fragment specific for IL-17C as disclosed in Table 1 and a pharmaceutically acceptable carrier or excipient.

An exemplary antibody or antibody fragment comprising a heavy chain comprising the variable heavy chain sequence of SEQ ID NO: 17 and a variable light chain comprising the amino acid sequence of SEQ ID NO: 16 is the fully human anti-IL-17C antibody known as MOR106.

In an embodiments, said antibody or antibody fragment is an antagonistic antibody or antibody fragment.

In certain embodiments, said antagonistic antibody or antibody fragment blocks the binding of IL-17C to IL-17RE.

In certain embodiments, said antagonistic antibody or antibody fragment specific for IL-17C antagonize any of the roles of IL-17C in AD.

In certain embodiments, the antibody or antibody fragment specific for IL-17C is an antibody or antibody fragment that specifically binds IL-17C.

In certain embodiments, said antibody or antibody fragment specific for IL-17C is an antibody or antibody fragment that specifically binds to human IL-17C.

In certain embodiments, said antibody or antibody fragment specific for IL-17C is an isolated monoclonal antibody or antibody fragment that specifically binds to human IL-17C.

In certain embodiments, said antibody or antibody fragment thereof specific for IL-17C is an isolated antibody or antibody fragment specific for IL-17C.

In certain embodiments, said antibody or antibody fragment thereof specific for IL-17C block the binding of IL-17C to IL-17RE.

In certain embodiments, said antibody or antibody fragment thereof specific for IL-17C blocks the binding of IL-17C to the receptor of IL-17C.

In certain embodiments, said antibody or antibody fragment thereof specific for IL-17C blocks the binding of IL-17C to the receptor of IL-17C, wherein said receptor is IL-17RE.

In certain embodiments, said antibody or antibody fragment thereof specific for IL-17C blocks the binding of IL-17C to IL-17RE.

In one embodiment said antibody or antibody fragment thereof is an IL-17C antagonist.

In one embodiment, said antibody or antibody fragment specific for IL-17C blocks the binding of IL-17C to one or more receptors of IL-17C.

Other antagonistic anti-IL-17C antibodies that can be used in the context of the methods of the present invention include any of the IL-17C specific antibodies as set forth in WO1999/060127, WO2013/057241, WO2017060289, and WO2017140831.

In other embodiments, the antibody or antibody fragment used in the present invention is an antibody or antibody fragment specific for a polypeptide encoding an amino acid sequence comprising SEQ ID NO: 1.

TABLE 1
Antibody sequences

| Antibody | | | |
|---|---|---|---|
| # | | SEQ ID NO: | [aa]/DNA |
| MOR106 | HCDR1 (Kabat) | SEQ ID NO: 7 | DYAMH |
| | HCDR2 (Kabat) | SEQ ID NO: 8 | YIGGVGEGTQYAESVKG |
| | HCDR3 (Kabat) | SEQ ID NO: 9 | GFAIRYYGFDY |
| | HCDR1 (Chothia) | SEQ ID NO: 10 | GFTVSDY |
| | HCDR2 (Chothia) | SEQ ID NO: 11 | GGVGEG |
| | HCDR3 (Chothia) | SEQ ID NO: 12 | GFAIRYYGFDY |
| | LCDR1 (Kabat& | SEQ ID NO: 13 | SGDKLGDKYAY |
| | Chothia) | | |
| | LCDR2 (Kabat & | SEQ ID NO: 14 | QDSKRPS |
| | Chothia) | | |
| | LCDR3 (Kabat & | SEQ ID NO: 15 | QVFTFPLVTT |
| | Chothia) | | |
| | VL | SEQ ID NO: 16 | SYELTQPPSVSVSPGQTASITCSGDKLGDKYA |
| | | | YWYQQKPGQSPVLVIYQDSKRPSGIPERFSG |
| | | | SNSGNTATLTISGTQAEDEADYYCQVFTFPLV |
| | | | TTVFGGGTKLTVLGQ |
| | VH | SEQ ID NO: 17 | EVQLLESGGGLVQPGGSLRLSCAASGFTVSD |
| | | | YAMHWVRQAPGKGLEWVSYIGGVGEGTQYA |
| | | | ESVKGRFTISRDNSKNTLYLQMNSLRAEDTAV |
| | | | YYCARGFAIRYYGFDYWGQGTLVTVSS |
| | VL (DNA) | SEQ ID NO: 18 | agctatgaactgacccagccgccgagcgttagcgttagcccag |
| | | | gccagaccgccagcattacctgtagcggcgacaaactgggcg |
| | | | acaaatacgcctactggtatcagcagaaaccgggccagagcc |
| | | | cggtgctggttatctatcaggatagcaaacgcccgagcggcatt |
| | | | ccagaacgctttagcggcagcaacagcggcaacaccgccac |
| | | | cctgaccattagcggcacccaggccgaagacgaagccgatta |
| | | | ttactgccaggttttcactttcccgctggttactactgtgtttggcggc |
| | | | ggtaccaagctgaccgtgctgggccag |
| | VH (DNA) | SEQ ID NO: 19 | gaagtgcagctgctggaaagcggtggcggtctggtgcagcca |
| | | | ggtggtagcctgcgcctgagctgtgccgcaagcggcttcacagt |
| | | | gtccgactacgcaatgcattgggtgcgccaagcaccaggcaa |
| | | | aggcctggaatgggtgagttacataggtggcgtgggtgagggg |
| | | | acacaatatgcagagagcgtgaaaggtcgctttaccattagtcg |
| | | | cgataacagcaaaaacaccctgtatctgcaaatgaacagcctg |
| | | | cgggcagaagataccgcagtttattattgcgcgcgtggtttcgca |
| | | | atccgttattatggatttgattattggggccagggcaccctggttac |
| | | | tgtctcgagc |
| | HCDR1 (Kabat) | SEQ ID NO: 33 | gactacgcaatgcat |
| | HCDR2 (Kabat) | SEQ ID NO: 34 | tacataggtggcgtgggtgaggggacacaatatgcagagagc |
| | | | gtgaaaggt |
| | HCDR3 (Kabat) | SEQ ID NO: 35 | ggtttcgcaatccgttattatggatttgattat |
| | HCDR1 (Chothia) | SEQ ID NO: 36 | ggcttcacagtgtccgactac |
| | HCDR2 (Chothia) | SEQ ID NO: 37 | ggtggcgtgggtgagggg |
| | HCDR3 (Chothia) | SEQ ID NO: 38 | ggtttcgcaatccgttattatggatttgattat |
| | LCDR1 (Kabat & | SEQ ID NO: 39 | agcggcgacaaactgggcgacaaatacgcctac |
| | Chothia) | | |
| | LCDR2 (Kabat & | SEQ ID NO: 40 | caggatagcaaacgcccgagc |
| | Chothia) | | |
| | LCDR3 (Kabat & | SEQ ID NO: 41 | caggttttcactttcccgctggttactact |
| | Chothia) | | |
| | Light chain | SEQ ID NO: 42 | SYELTQPPSVSVSPGQTASITCSGDKLGDKYA |
| | | | YWYQQKPGQSPVLVIYQDSKRPSGIPERFSG |
| | | | SNSGNTATLTISGTQAEDEADYYCQVFTFPLV |
| | | | TTVFGGGTKLTVLGQPKAAPSVTLFPPSSEEL |
| | | | QANKATLVCLISDFYPGAVTVAWKADSSPVKA |
| | | | GVETTTPSKQSNNKYAASSYLSLTPEQWKSH |
| | | | RSYSCQVTHEGSTVEKTVAPTECS |
| | Heavy chain | SEQ ID NO: 43 | EVQLLESGGGLVQPGGSLRLSCAASGFTVSD |
| | (IgG1) | | YAMHWVRQAPGKGLEWVSYIGGVGEGTQYA |
| | | | ESVKGRFTISRDNSKNTLYLQMNSLRAEDTAV |
| | | | YYCARGFAIRYYGFDYWGQGTLVTVSSASTK |
| | | | GPSVFPLAPSSKSTSGGTAALGCLVKDYFPEP |
| | | | VTVSWNSGALTSGVHTFPAVLQSSGLYSLSS |
| | | | VVTVPSSSLGTQTYICNVNHKPSNTKVDKRVE |
| | | | PKSCDKTHTCPPCPAPELLGGPSVFLFPPKPK |
| | | | DTLMISRTPEVTCVVVDVSHEDPEVKFNWYV |
| | | | DGVEVHNAKTKPREEQYNSTYRVVSVLTVLH |
| | | | QDWLNGKEYKCKVSNKALPAPIEKTISKAKGQ |
| | | | PREPQVYTLPPSREEMTKNQVSLTCLVKGFY |
| | | | PSDIAVEWESNGQPENNYKTTPPVLDSDGSF |
| | | | FLYSKLTVDKSRWQQGNVFSCSVMHEALHN |
| | | | HYTQKSLSLSPGK |
| | Light chain | SEQ ID NO: 44 | agctatgaactgacccagccgccgagcgttagcgttagcccag |
| | (DNA) | | gccagaccgccagcattacctgtagcggcgacaaactgggcg |
| | | | acaaatacgcctactggtatcagcagaaaccgggccagagcc |
| | | | cggtgctggttatctatcaggatagcaaacgcccgagcggcatt |
| | | | ccagaacgctttagcggcagcaacagcggcaacaccgccac |
| | | | cctgaccattagcggcacccaggccgaagacgaagccgatta |
| | | | ttactgccaggttttcactttcccgctggttactactgtgtttggcggc |
| | | | ggtaccaagctgaccgtgctgggccagcccaaagccgcccct |
| | | | agcgtgaccctgttccccccaagcagcgaggaactccaggcc |
| | | | aacaaggccaccctcgtgtgcctgatcagcgacttctaccctgg |
| | | | cgccgtgaccgtggcctggaaggccgatagcagccctgtgaa |
| | | | ggccggcgtggaaaccaccacccccagcaagcagagcaac |
| | | | aacaaatacgccgccagcagctacctgagcctgacccccgag |
| | | | cagtggaagtcccacagatcctacagctgccaggtcacacac |
| | | | gagggcagcaccgtggaaaagaccgtggcccccaccgagtg |
| | | | cagc |
| | Heavy chain | SEQ ID NO: 45 | gaagtgcagctgctggaaagcggtggcggtctggtgcagcca |
| | (IgG1, DNA) | | ggtggtagcctgcgcctgagctgtgccgcaagcggcttcacagt |
| | | | gtccgactacgcaatgcattgggtgcgccaagcaccaggcaa |
| | | | aggcctggaatgggtgagttacataggtggcgtgggtgagggg |
| | | | acacaatatgcagagagcgtgaaaggtcgctttaccattagtcg |
| | | | cgataacagcaaaaacaccctgtatctgcaaatgaacagcctg |
| | | | cgggcagaagataccgcagtttattattgcgcgcgtggtttcgca |
| | | | atccgttattatggatttgattattggggccagggcaccctggttac |
| | | | tgtctcgagcgcgtcgaccaaaggccccagcgtgttccctctgg |
| | | | cccccagcagcaagagcacctctggcggaacagccgccctg |
| | | | ggctgcctggtcaaggactacttccccgagcccgtgaccgtgtc |
| | | | ctggaactctggcgccctgaccagcggcgtgcacacctttccag |
| | | | ccgtgctccagagcagcggcctgtacagcctgagcagcgtcgt |
| | | | gaccgtgcccagcagcagcctgggcacccagacctacatctg |
| | | | caacgtgaaccacaagcccagcaacacaaaggtggacaag |
| | | | cgggtggaacccaagagctgcgacaagacccacacctgtccc |
| | | | ccctgccctgcccctgaactgctgggaggcccctccgtgttcctg |
| | | | ttccccccaaagcctaaggacaccctgatgatcagccggaccc |
| | | | ccgaagtgacctgcgtggtggtggacgtgtcccacgaggaccc |
| | | | tgaagtgaagtttaattggtacgtggacggcgtggaagtgcaca |
| | | | acgccaagaccaagcccagagaggaacagtacaacagcac |
| | | | ctaccgggtggtgtccgtgctgaccgtgctgcaccaggactggc |
| | | | tgaacggcaaagagtacaagtgcaaggtgtccaacaaggcc |
| | | | ctgcctgcccccatcgagaaaaccatcagcaaggccaaaggc |
| | | | cagccccgcgagccccaggtgtacacactgccccctagccgg |
| | | | gaagagatgaccaagaaccaggtgtccctgacctgcctcgtga |
| | | | agggcttctaccccagcgacattgccgtggaatgggagagcaa |
| | | | cggccagcccgagaacaactacaagaccaccccccctgtgct |
| | | | ggacagcgacggctcattcttcctgtacagcaagctgaccgtgg |
| | | | acaagagccggtggcagcagggcaacgtgttcagctgctccgt |
| | | | gatgcacgaggccctgcacaaccactacacccagaagtccct |
| | | | gagcctgagccccggcaag |
| | DNA (optimized) | | |
| | HCDR1 (Kabat) | SEQ ID NO: 58 | gactacgctatgcac |
| | HCDR2 (Kabat) | SEQ ID NO: 59 | tatatcggcggcgtgggcgagggcacccagtacgctgagtctgt |
| | | | gaagggc |
| | HCDR3 (Kabat) | SEQ ID NO: 60 | ggcttcgccatccggtactacggcttcgactac |
| | HCDR1 (Chothia) | SEQ ID NO: 61 | ggcttcaccgtgtccgactac |
| | HCDR2 (Chothia) | SEQ ID NO: 62 | ggcggcgtgggcgagggc |
| | HCDR3 (Chothia) | SEQ ID NO: 63 | ggcttcgccatccggtactacggcttcgactac |
| | LCDR1 (Kabat & | SEQ ID NO: 64 | tccggcgacaagctgggcgataagtacgcctac |
| | Chothia) | | |
| | LCDR2 (Kabat & | SEQ ID NO: 65 | caggactccaagcggccctcc |
| | Chothia) | | |
| | LCDR3 (Kabat & | SEQ ID NO: 66 | caggtgttcaccttccccctggtcaccacc |
| | Chothia) | | |
| | VL (DNA) | SEQ ID NO: 67 | tcctacgagctgacccagcccccctccgtgtccgtgtctcctggc |
| | | | cagaccgcctccatcacctgttccggcgacaagctgggcgata |
| | | | agtacgoctactggtatcagcagaagcccggccagtcccccgt |
| | | | gctggtcatctaccaggactccaagcggccctccggcatccctg |
| | | | agcggttctccggctccaactccggcaacaccgccaccctgac |
| | | | catctccggcacccaggccgaggacgaggccgactactactg |
| | | | ccaggtgttcaccttccccctggtcaccaccgtgttcggcggagg |
| | | | caccaagctgaccgtgctgggccag |
| | VH (DNA) | SEQ ID NO: 68 | gaggtgcagctgctggaatccggcggaggactggtgcagcct |
| | | | ggcggctccctgagactgtcttgcgccgcctccggcttcaccgtg |
| | | | tccgactacgctatgcactgggtccgacaggcccctggcaagg |
| | | | gcctggaatgggtgtcctatatcggcggcgtgggcgagggcac |
| | | | ccagtacgctgagtctgtgaagggccggttcaccatctcccggg |
| | | | acaactccaagaacaccctgtacctgcagatgaactccctgcg |
| | | | ggccgaggacaccgccgtgtactactgtgccagaggcttcgcc |
| | | | atccggtactacggcttcgactactggggccagggcaccctggt |
| | | | caccgtgtctagc |
| | Light chain | SEQ ID NO: 69 | tcctacgagctgacccagcccccctccgtgtccgtgtctcctggc |
| | (DNA) | | cagaccgcctccatcacctgttccggcgacaagctgggcgata |
| | | | agtacgcctactggtatcagcagaagcccggccagtcccccgt |
| | | | gctggtcatctaccaggactccaagcggccctccggcatccctg |
| | | | agcggttctccggctccaactccggcaacaccgccaccctgac |
| | | | catctccggcacccaggccgaggacgaggccgactactactg |
| | | | ccaggtgttcaccttccccctggtcaccaccgtgttcggcggagg |
| | | | caccaagctgaccgtgctgggccagcctaaggccgctccctcc |
| | | | gtgaccctgttccccccatcctccgaggaactgcaggccaaca |
| | | | aggccaccctggtctgcctgatctccgacttctaccctggcgccg |
| | | | tgaccgtggcctggaaggccgacagctctcctgtgaaggccgg |
| | | | cgtggaaaccaccaccccctccaagcagtccaacaacaaata |
| | | | cgccgcctcctcctacctgtccctgacccccgagcagtggaagt |
| | | | cccaccggtcctacagctgccaggtcacacacgagggctcca |
| | | | ccgtggaaaagaccgtggcccctaccgagtgctcc |
| | Heavy chain | SEQ ID NO: 70 | gaggtgcagctgctggaatccggcggaggactggtgcagcct |
| | (IgG1, DNA) | | ggcggctccctgagactgtcttgcgccgcctccggcttcaccgtg |
| | | | tccgactacgctatgcactgggtccgacaggcccctggcaagg |
| | | | gcctggaatgggtgtcctatatcggcggcgtgggcgagggcac |
| | | | ccagtacgctgagtctgtgaagggccggttcaccatctcccggg |
| | | | acaactccaagaacaccctgtacctgcagatgaactccctgcg |
| | | | ggccgaggacaccgccgtgtactactgtgccagaggcttcgcc |
| | | | atccggtactacggcttcgactactggggccagggcaccctggt |
| | | | caccgtgtctagcgcctccaccaagggcccctccgtgttccctct |
| | | | ggccccctccagcaagtccacctctggcggcaccgctgccctg |
| | | | ggctgcctggtcaaggactacttccccgagcccgtgaccgtgtc |
| | | | ctggaactctggcgccctgacctccggcgtgcacaccttccctg |
| | | | ccgtgctgcagtcctccggcctgtactccctgtcctccgtcgtgac |
| | | | cgtgccctccagctctctgggcacccagacctacatctgcaacg |
| | | | tgaaccacaagccctccaacaccaaggtggacaagcgggtg |
| | | | gaacccaagtcctgcgacaagacccacacctgtcccccctgcc |
| | | | ctgcccctgaactgctgggcggaccttccgtgttcctgttcccccc |
| | | | aaagcccaaggacaccctgatgatctcccggacccccgaagt |
| | | | gacctgcgtggtggtggacgtgtcccacgaggaccctgaagtg |
| | | | aagttcaattggtacgtggacggcgtggaagtgcacaacgcca |
| | | | agaccaagcccagagaggaacagtacaactccacctaccgg |
| | | | gtggtgtccgtgctgaccgtgctgcaccaggactggctgaacgg |
| | | | caaagagtacaagtgcaaggtgtccaacaaggccctgcctgc |
| | | | ccccatcgaaaagaccatctccaaggccaagggccagcccc |
| | | | gcgagccccaggtgtacacactgccccctagccgggaagag |
| | | | atgaccaagaaccaggtgtccctgacctgtctggtcaagggctt |
| | | | ctacccctccgacattgccgtggaatgggagtccaacggccag |
| | | | cccgagaacaactacaagaccaccccccctgtgctggactcc |
| | | | gacggctcattcttcctgtactccaagctgaccgtggacaagtcc |
| | | | cggtggcagcagggcaacgtgttctcctgctccgtgatgcacga |
| | | | ggccctgcacaaccactacacccagaagtccctgtccctgagc |
| | | | cccggcaag |

### Examples

### Example 1—Background of the Phase/Study

The exemplary antagonistic antibody specific for IL-17C used in the following examples is the human antibody MOR106, comprising a HCDR1 region of amino acid sequence SEQ ID NO: 7, a HCDR2 region of amino acid sequence SEQ ID NO: 8, a HCDR3 region of amino acid sequence SEQ ID NO: 9, a LCDR1 region of amino acid sequence SEQ ID NO: 13, a LCDR2 region of amino acid sequence SEQ ID NO: 14 and a LCDR3 region of amino acid sequence SEQ ID NO: 15.

MOR106 was studied in a randomized, double-blind, placebo-controlled Phase 1 trial, evaluating single ascending doses of the molecule in healthy volunteers (SAD part of the trial), and multiple ascending doses in patients with moderate-to-severe atopic dermatitis (MAD part of the trial).

The primary objective of the Phase 1 study was to evaluate the safety and tolerability of MOR106. The study's secondary objective was to characterize the pharmacokinetic profile of MOR106 in healthy volunteers and patients. The (MAD part of the) trial was not powered for efficacy. Nonetheless efficacy was an exploratory endpoint in the MAD

### Example 2—MAD

The MAD part of the Phase I study explored the repeated dose treatment of patients with moderate-to-severe atopic dermatitis with intravenously administered MOR106. This part of the study was a 99 days, double-blind, randomized, placebo-controlled, (sequential) multiple ascending repeated dose study to assess the safety and tolerability of administered MOR106 in patients with atopic dermatitis.

The overall study duration was 14 weeks. The treatment period was 4 weeks (D1 to 29), with four weekly intravenous infusions on days 1, 8, 15 and 22, respectively. The end of the treatment period study was on day 29 (week 4).

After the end of the treatment period patients were followed for 10 weeks (70 days) (days 30 to 99).

In the MAD part, 25 patients were included and randomized in a 3:1 ratio to receive MOR106 or placebo in each of the three ascending dose cohorts (overall 6 subjects were treated with placebo, and 19 subjects treated with MOR106 (7 in cohort 1 (1 mg/kg), 6 in cohort 2 (4 mg/kg) and 6 in cohort 3 (10 mg/kg cohort)).

2 subjects in the MAD, cohort 1, withdrew from the study (one treated with the active drug (1 mg/kg and one subject treated with placebo). Overall, 6 of 25 patients discontinued the study comprising 3 subjects treated with the drug and 3 subjects treated with placebo. 3 discontinuations occurred during the treatment period and 3 discontinuations occurred during the follow-up phase.

TABLE 2
Discontinuation Summary (MAD)

| Cohort | MOR106/PBO | Day | Reason |
|---|---|---|---|
| 1 mg/kg | MOR106 | 22 | Dermatitis atopic |
| 1 mg/kg | MOR106 | 43 | Withdrawal by subject |
| 1 mg/kg | Placebo | 28 | Withdrawal by subject |
| 1 mg/kg | Placebo | 65 | Prohibited medication |
| 10 mg/kg | MOR106 | 59 | Prohibited medication |
| 10 mg/kg | Placebo | 27 | Dermatitis atopic |

### Patient (MAD) Inclusion Criteria

(1) Male or female between 18-65 years of age (extremes included)
(2) A BMI between 18-30 kg/m²
(3) Diagnosis of Atopic Dermatitis (AD) for at least 6 months as per the Hanifin and Rajka Criteria.
(4) EASI≥16 at the screening and baseline visits.
(5) IGA score≥3 (on the 0 to 4 IGA scale, in which 3 is moderate and 4 is severe) at the screening and baseline visits.
(6) Greater than or equal to 10% body surface area (BSA) of AD involvement at screening.
(7) Willingness to continue stable use of an additive free, basic, bland emollient twice daily for at least 7 days before the baseline visit.
(8) Subject is a candidate for systemic therapy and is not responding adequately or has a contraindication to topical corticosteroids and/or topical calcineurin inhibitors (per Investigator's judgement).
(9) Absence of current active, latent or history of tuberculosis (TB) infection based on medical history and as determined by a negative QuantiFERON TB Gold test at screening.

### Patient (MAD) Exclusion Criteria:

(1) Active skin infection requiring systemic antibiotics at baseline, or have clinical signs of infective eczema within 1 week before baseline
(2) Having used any of the following treatments within 4 weeks before the baseline visit
   i. Immunosuppressive/immunomodulating drugs (e.g., systemic corticosteroids, cyclosporine, mycophenolate-mofetil, IFN-γ, azathioprine, methotrexate, etc.)
   ii. Phototherapy (UVB or PUVA) for AD
(3) Treatment with topical corticosteroids (TCS) or topical calcineurin inhibitors (TCI) within 2 weeks before the baseline visit.
(4) Treatment with biologics within 5 half-lives (if known) or 12 weeks prior to baseline visit, whichever is longer.
(5) Regular use (more than 2 visits per week) of a tanning booth/parlor within 4 weeks of the screening visit.
(6) Regular daily use of oral nonsteroidal anti-inflammatory drugs (NSAIDs), except low-dose aspirin (≤200 mg/day) for cardioprotection, within 7 days prior to screening.
(7) Active chronic or acute infection requiring treatment with systemic antibiotics, antivirals, antiparasitics, antiprotozoals, or antifungals within 4 weeks before the baseline visit
(8) Known or suspected history of immunosuppression, including history of invasive opportunistic infections
(9) Female subjects must have a negative serum pregnancy test at screening and a negative urine pregnancy test at baseline.

The primary endpoint of the study was to monitor incidences of treatment-emergent adverse events (TEAEs). The efficacy measure was a purely exploratory endpoint and the study was not powered for efficacy.

Severity and degree of the diseases can be determined by various commonly used methods as further described in the following.

Eczema Area and Severity Index (EASI): The EASI is a validated measure used in clinical practice and clinical studies to assess the severity and extent of atopic dermatitis (Hanifin et al 2001, Exp. Dermatol. 10: 1 1-18). Four atopic dermatitis disease characteristics will be assessed for severity on a scale of “0” (absent) through “3” (severe). In addition, the area of atopic dermatitis involvement will be assessed as a percentage by body area of head, trunk, arms, and legs, and converted to a score of 0 to 6. The EASI score ranges are between 0 and 72.

SCORAD: The SCORAD is a validated tool used in clinical research and clinical practice that was developed to standardize the evaluation of the extent and severity of atopic dermatitis (Dermatology, vol. 186(1), pp. 23-31, 1993). The extent of atopic dermatitis is assessed as a percentage of each defined body area and reported as the sum of all areas, with a maximum score of 100% (assigned as “A” in the overall SCORAD calculation). The severity of 6 specific symptoms of atopic dermatitis is assessed using the following scale: none (0), mild (1), moderate (2), or severe (3) (for a maximum of 18 total points, assigned as “B” in the overall SCORAD calculation). Subjective assessment of itch and sleeplessness is recorded for each symptom by the subject or relative on a visual analogue scale, where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A/5+7B/2+C and ranges between 0 and 133.

Investigator's Global Assessment (IGA): The IGA score was assessed at every clinic visit. The IGA is an assessment scale used in clinical studies to determine severity of atopic dermatitis and clinical response to treatment based on a 5-point scale ranging from 0 (clear) to 4 (severe).

A decrease of any of the aforementioned scores, i.e. the Eczema Area and Severity Index (EASI) score, the SCORAD score or the Investigator's Global Assessment (IGA) score indicates that the treatment seems to be effective.

The patient populations of the MAD study is characterized as follows.

TABLE 3
Patient disposition and baseline characteristics (MAD)

| Baseline Characteristics | ITT* population (n = 25) |
|---|---|
| Age, years, mean | 33.8 |
| Range | 19-64 |
| Height,cm, mean | 168.54 |
| Range | 154.0-189.5 |
| Weight, kg, mean | 68.93 |
| Range | 49.5-104.0 |
| BMI | 24.13 |
| range | 18.4-29.9 |
| Male, n (%) | 12 (48) |
| Female, n (%) | 13 (52) |

*all randomized subjects who have at least received one dose of study drug and have at least one post-BL efficacy assessment.

TABLE 4
Mean Baseline EASI Characteristics

| | 1 mg/kg | 4 mg/kg | 10 mg/kg | Placebo |
|---|---|---|---|---|
| Mean | 37.40 | 33.92 | 34.08 | 33.74 |
| Median | 38.90 | 36.55 | 31.60 | 30.30 |
| Min, max | 22.8-44.0 | 18.1-47.4 | 16.4-55.0 | 19.4-55.2 |

### Example 3—Treatment with the Study Drug

MOR106 was stored under at 2 to 8° C.

The study medication was prepared by the unblinded pharmacist by adding MOR106 into the infusion container, resulting in an intravenous solution with the respective concentration of the planned dose level. The 0.9% sodium chloride infusion container will be used as placebo in the proposed clinical study. Instructions for the preparation of the various concentrations of MOR106 solution for intravenous infusion and recommended pump speed and duration of infusion was described in a Pharmacy Manual. All dosing was carried out by the Investigator or by a member of the clinical center designated by the Investigator.

For the preparation and administration of MOR106 by intravenous infusion, the following components were supplied to the clinical centers:
Vials containing 20 mg/ml MOR106 injectable solution, buffered at a pH 5.5.
0.9% sodium chloride injectable solution in infusion container.

The drug was dispensed intravenously using a validated infusion pump. The infusion bag was blinded to make sure that placebo cannot be distinguished from the active drug due to color and aspect differences.

Subject were monitored for at least 6 hours following the completion of the first two doses (Day 1, 8) and for at least 4 hours for the subsequent doses (Day 15, 22).

The Phase I-unit pharmacist or delegate prepared the actual dosages according to the randomization schedule before the administrations. The weight determined at screening was used to calculate the applicable dose. Detailed instructions for aseptic preparation of the applicable doses were provided by the Sponsor in a Pharmacy Manual.

### Example 4—Safety Assessment

The safety assessment was based on Adverse Events, physical examinations, vital signs, 12-lead ECG and safety laboratory assessments.

Adverse Events (AEs) including Serious Adverse Events (SAEs) were recorded from signature of the Informed Consent Form (ICF) until the final follow-up visit or until 30 days after last study drug administration (whichever comes last).

Research samples (e.g. serum/plasma) were collected at day 1/baseline, and days 8, 15, 22, 29, 57, 85, and 99 (end of study) or early termination.

An Adverse Event (AE) is any untoward medical occurrence in a subject or clinical investigation subject administered a pharmaceutical product.

An AE can, therefore, be any unfavorable and unintended sign (including abnormal laboratory finding), symptom, or disease temporally associated with the use of a medicinal product, whether or not considered related to the medicinal (investigational) product.

AEs also include: any worsening (i.e., any clinically significant change in frequency and/or intensity) of a pre-existing condition that is temporally associated with the use of the study drug; abnormal laboratory findings considered by the Investigator to be clinically significant; and any untoward medical occurrence.

A treatment emergent adverse event (TEAE) is an adverse event that occurs only once treatment has started.

A Serious Adverse Event (SAE) is any untoward medical occurrence that at any dose results in death; is life-threatening; requires in-patient hospitalization or prolongation of existing hospitalization; results in persistent or significant disability/incapacity; is a congenital anomaly/birth defect; or is an important medical event.

Infusion Related Reactions (IRR) to monoclonal antibodies typically develop within 30 minutes to two hours after the initiation of the drug infusion, although symptoms may be delayed up to 24 hours. Therefore, IRRs were defined as adverse events of special interest for the Phase I trial.

### Outcome Safety Assessment of the MAD Part of the Phase/Trial:

Intravenous administration of MOR106 to patients with moderate-to-severe AD was generally safe and well-tolerated at all doses tested.

No SAE (including deaths) or IRR were reported. Adverse events observed were mild to moderate and transient in nature.

Treatment Emergent Adverse Events (TEAEs) reported by >=2 subjects over 14 weeks are summarized in Table 5.

Skin related adverse events over 14 weeks are summarized in Table 6.

TABLE 5
MAD: Treatment Emergent Adverse Events >= 2 subjects over 14 weeks

| MedDRA * | Cohort | Cohort | Cohort | |
|---|---|---|---|---|
| preferred term | MOR106 | MOR106 | MOR106 | |
| (n, %) | 1 mg/kg | 4 mg/kg | 10 mg/kg | Placebo |
| Dermatitis | 1 (16.7) | 1 (16.7) | | 2 (28.6) |
| atopic | | | | |
| Blister | 1 (16.7) | 1 (16.7) | | |
| Nasopharyngitis | 2 (33.3) | 2 (33.3) | | 1 (14.3) |
| Blood Creatin | | 1 (16.7) | 1 (16.7) | |
| phospho kinase | | | | |
| (CPK) increased | | | | |

*Medical Dictionary for Regulatory Activities

TABLE 6
First in human MAD - Skin related TEAE (Treatment
Emergent Adverse Events) over 14 weeks

| | No. of | Cohort (No. | | |
|---|---|---|---|---|
| MedDRA | subjects | subjects per | Relation- | |
| preferred term | (n, %) | cohort) | ship | Intensity |
| Dermatits | 1 (16,7) | 1 mg/kg | Possible | Mild |
| acneiform | | | | |
| Dermatitis | 1 (16,7) | 1 mg/kg | Not related | Moderate |
| atopic | 1 (16,7) | 4 mg/kg | Not related | Moderate |
| | 2 (28.6%) | Placebo | Not related | Moderate |
| Blisters | 1 (16,7) | 1 mg/kg (1) | Probable | Moderate |
| | 1 (16,7) | 4 mg/kg (1) | Unlikely | Mild |
| Skin fissures | 1 (16,7) | 10 mg/kg (1) | Possible | Moderate |

### Example 5—Efficacy

Data Analysis: Efficacy data was analyzed using descriptive statistics of actual values, changes from baseline, and percent changes from baseline. MOR106 and placebo were compared using a Kruskal-Wallis test (overall treatment effect) and Wilcoxon rank sum tests (pairwise comparisons versus placebo). Within-group comparisons of each visit versus baseline were investigated using a Wilcoxon signed rank test. And an exploratory monotone dose-response trend was assessed using a Jonckheere-Terpstra test.

Analysis of Pharmacokinetics: The terminal elimination half-life was determined using non-compartmental data analysis (NCA).

Surprisingly, although the study was not powered for efficacy, the results do indicate that the study drug is efficacious even at very low doses such at a dose of 1 mg/kg.

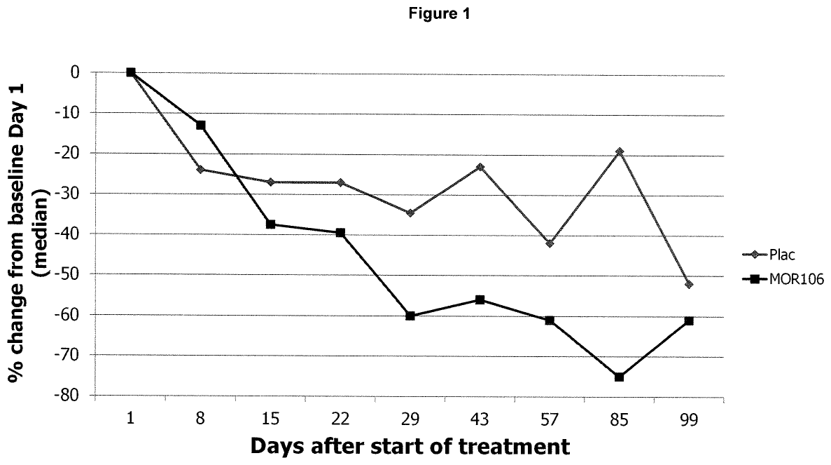

The efficacy results obtained from the study are summarized in FIGS. 1-5. As noted above, patients were treated with MOR106 with either 1 mg/kg, 4 mg/kg or 10 mg/kg intravenously once a week for 4 weeks, or with placebo

MOR106 was effective in patients with moderate-to-severe AD and administration of MOR106 significantly improved AD disease activity and severity.

### EASI Results

Analysis of the median percentage reduction form baseline of pooled EASI patients (1 mg/kg, 4 mg/kg and 10 mg/kg cohort) over the time course of the study revealed significant improvements over placebo starting from day 15 of the study (within treatment course) with more patients had 50% percentage reduction from baseline at day 29 (1 week after last dose) and thereafter until the end of the study at day 99 (FIG. 1).

This data clearly indicates that MOR106 is efficacious even 10 weeks post-treatment without (re)-worsening in EASI parameters.

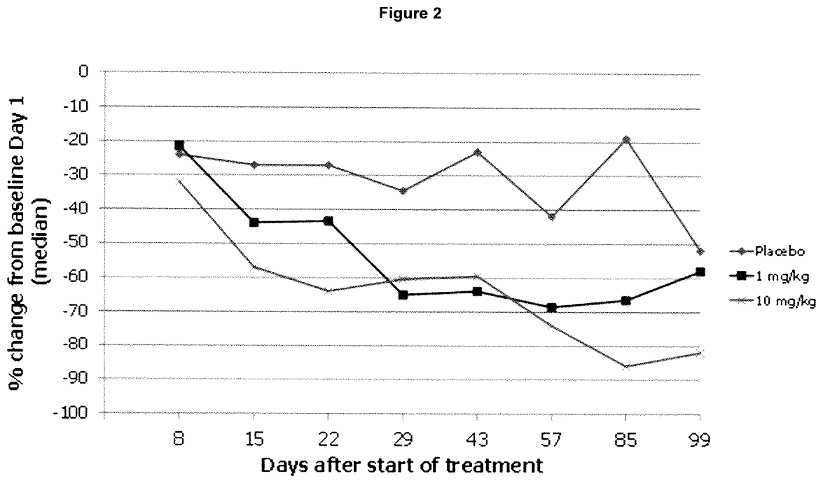

Moreover, analysis of the median percentage reduction form baseline of EASI patients of the 1 mg/kg cohort vs. patients from the 10 mg/kg cohort clearly revealed that administration of 1 mg/kg of MOR106 is similar efficacious at day 29 though day 57 (2 to 3 weeks after last treatment) (FIG. 2).

The finding that MOR106 is efficacious at a dose of 1 mg/kg even in the post-treatment phase is even more surprising considering the fact that for effective treatment of AD it is for important for an antibody such like MOR106 to diffuse from the blood stream to the affected skin keratinocytes in the epidermis.

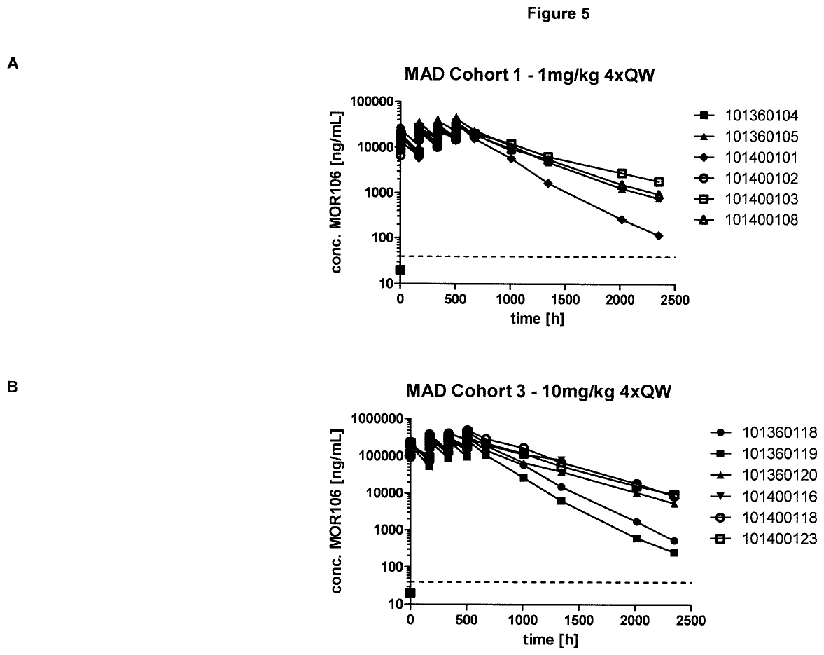

Based on an estimated diffusion rate of ˜ 16% (Shah et al. 2013), which means that only 16% of the antibody present in serum actually reaches the affected cells after intravenous administration, the continuous IL-17C production and considering patient heterogeneity, it appears that a minimum clinical effect serum level in AD patients can be anticipated to be at a serum concentration of approximately 100 ng/ml antibody (see FIG. 5A, MOR106 serum levels for patient 101400101 at day 99).

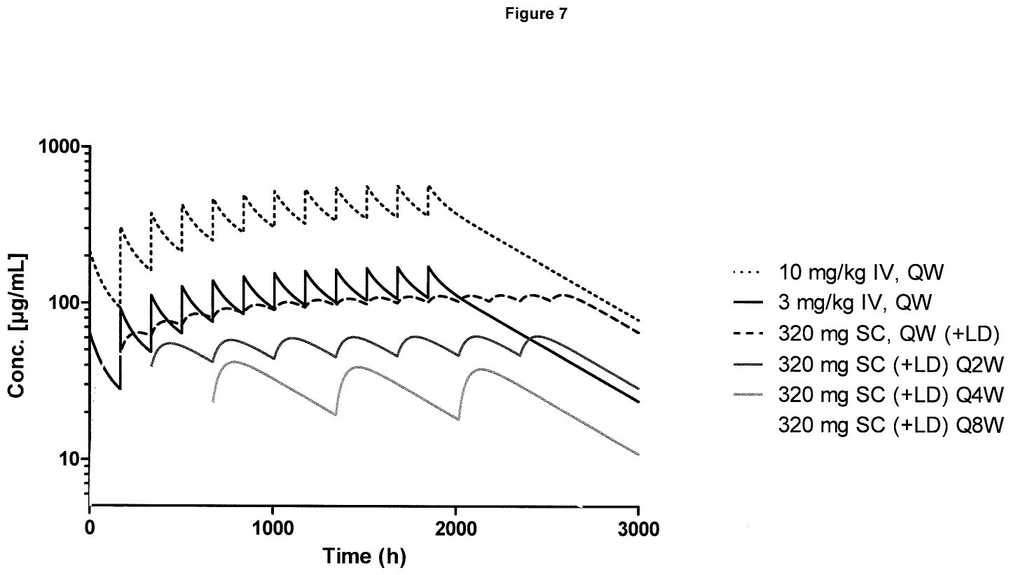

FIG. 7 indicates simulated serum levels of MOR106 over the course of the Phase 2 clinical trial in Example 6. As it can be deduced from the simulation curves, the intravenous weekly administration of 3 mg/kg MOR106 results in comparable drug serum levels as weekly subcutaneous fixed doses of 320 mg antibody including a first loading dose of 640 mg.

Consequently, it can be expected that administering MOR106 subcutaneously at a dose of 320 mg or at least at a dose which is equivalent to a dose of 1 mg/kg intravenously will be efficacious in the treatment of AD patients.

### SCORAD Results

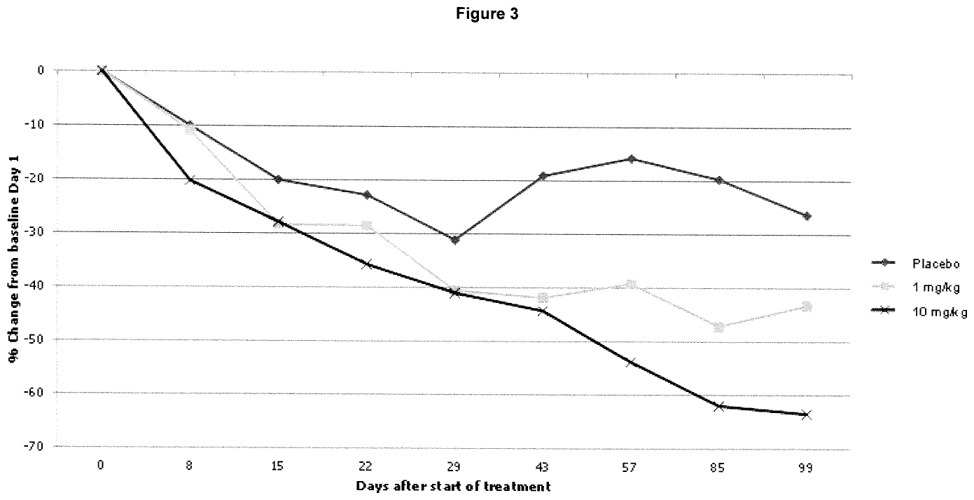

At day 29 (1 week after last treatment), administration of 1 mg/kg and 10 mg/kg of MOR106 achieved significant improvements vs. placebo of mean percentage reduction from baseline in SCORAD with more patients had 40% mean percentage reduction from baseline at day 29. Moreover, administration of 1 mg/kg of MOR106 appeared to be similar efficacious as administrating of 10 mg/kg of MOR106 at day 15 (during treatment course) as well as at day 29 and day 43 (2 and 4 weeks after last treatment) (FIG. 3).

These data clearly indicate the surprising finding that MOR106 is efficacious even at a low dose of 1 mg/kg up to 2 weeks post-treatment.

### IGA Results

All subjects entered the study with IGA 3 or 4 (moderate to severe atopic dermatitis).

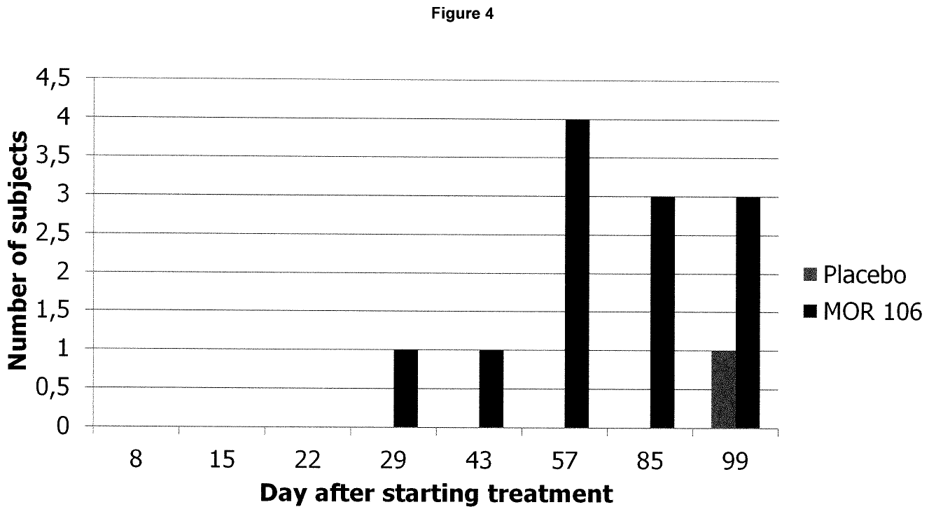

Patients treated with MOR106 (pooled subjects: 1 mg/kg, 4 mg/kg, 10 mg/kg) achieved IGA=1 with MOR106 at several time-points such as on day 29, 43, 57, 85, and day 99; whereas only 1 patient achieved IGA=1 with placebo at one day (day 99) (FIG. 4).

These data demonstrate that administration of MOR106 is efficacious in improving IGA parameters up to 10 weeks post-treatment.

PK Results

The determined terminal elimination half-life of MOR106 in individual patients administered with 1 mg/kg or 10 mg/kg MOR106 was in the range of 12-17 days (FIG. 5A and FIG. 5B, respectively).

In general, serum levels of MOR106 were dose-dependent.

Serum levels of MOR106 during the treatment course (day 1 to day 29) varied between 6.000 ng/mL and 42.000 ng/mL within the 1 mg/kg cohort.

At day 99, (last day of the study, post-treatment phase), serum levels of MOR106 were in the range of 100-2000 ng/mL in the 1 mg/kg cohort and 250-9.500 ng/mL in the 10 mg/kg cohort.

### Example 6—Phase 2 Clinical Trial with a Subcutaneous and an Intravenous Formulation of MOR0106

### A MOR106 Subcutaneous Dose Range Finding Study is Conducted

The clinical trial set our herein above is repeated in a Phase 2a clinical trial with a larger patient population to allow for a statistical significant efficacy assessment. AD subjects are administered 3 mg/kg or 10 mg/kg of MOR106, or placebo weekly (12 weekly doses). All administrations are intravenously.

The 10 mg/kg cohort is set-up to assess the maximum achievable efficacy in patients with AD, whereas the 3 mg/kg cohort shall simulate the maximum achievable efficacy in patients receiving a dose which corresponds to the approx. maximum drug amount which can be administered in a single (flat) subcutaneous dose to a patient (e.g. 320 mg of drug).

The phase 2b part is set-up to as a 16 week treatment course with 16 weekly doses to identify the optimal dose range for MOR106 for a subcutaneous applications. The study comprises 4 arms to evaluate a broad range of exposures with overlapping drug levels.

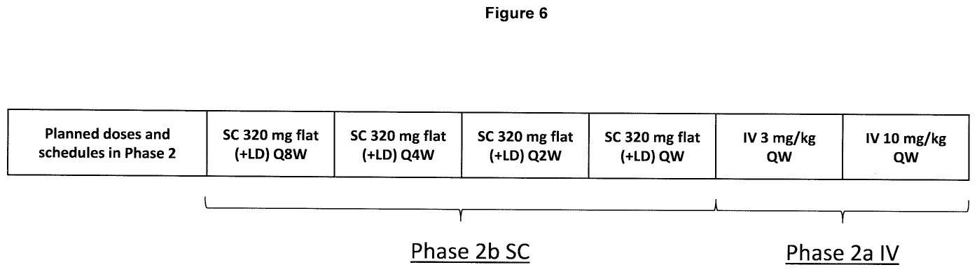

The first arm studies the weekly subcutaneous administration of 320 mg of MOR106 (including a first loading dose of 640 mg drug) to achieve the maximum subcutaneous drug amount possible. The systemic drug amount over 2 month in this arms corresponds to the systemic drug amount achievable by administering 3 mg/kg intravenously on a weekly basis (considering a 80 kg patient and an approximately 60% bioavailability (see also FIG. 6).

The second arm studies the subcutaneous administration of 320 mg of MOR106 once every two weeks (including a first loading dose of 640 mg drug) to achieve approximately the half-maximum subcutaneous drug amount possible.

The third arm studies the subcutaneous administration of 320 mg of MOR106 once every 4 weeks (including a first loading dose of 640 mg drug) to achieve approximately a third of the maximum subcutaneous drug amount possible.

The fourth arm studies the subcutaneous administration of 320 mg of MOR106 once every eight weeks (including a first loading dose of 640 mg drug) to achieve approximately one fifth of the maximum subcutaneous drug amount possible.

FIG. 7 indicates simulated serum levels of MOR106 over the course of the Phase 2 study using results from a Phase 1 study analyzed using a population PK approach and assuming a bioavailability of approximately 60% and an absorption rate constant of 0.01 h¹ for subcutaneous dosing. Various dosing and dosing schedules are simulated using the subcutaneous delivery of MOR106 in order to produce a serum concentrations that are similar those obtained after intravenous administration of 3 mg/kg, 1 mg/kg and below. These studies indicate that subcutaneous doses of 320 mg (including a first loading dose of 640 mg) can produce serum concentration similar to 3 mg/kg and 1 mg/kg, intravenously depending on the dosing frequency.

### Outcome of the Phase 2 Clinical Trial:

All treatment arms show a favorable safety profile and demonstrate clinical efficacy as measured by e.g., EASI, SCORAD and IGA scores.

1: A method for treatment of atopic dermatitis, said method comprising administering a pharmaceutical composition comprising an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15, wherein said antibody or antibody fragment is administered at a dose of 0.1 mg/kg-5 mg/kg.

2: The method of claim 1, wherein the antibody or antibody fragment is administered at a dose of 3 mg/kg or less.

3: The method of claim 1, wherein the antibody or antibody fragment is administered intravenously.

4: The method of claim 1, wherein said antibody or antibody fragment is administered at a first dose followed by one or more secondary doses, and wherein each of said first dose and each of said secondary doses comprise a dose of 3 mg/kg or less.

5: The method of claim 1, wherein said antibody or antibody fragment is administered subcutaneously.

6: The method of claim 5, wherein said antibody or antibody fragment is administered subcutaneously at a dose of 100 mg-400 mg.

7: The method of claim 6, wherein said antibody or antibody fragment is administered subcutaneously at a dose of 320 mg or less.

8: The method of claim 5, wherein prior to said administration additionally a loading dose of 200 mg-800 mg of said antibody or antibody fragment is administered.

9: The method of claim 8, wherein said loading dose is a loading dose of 640 mg or less.

10: The method of claim 5, wherein said antibody or antibody fragment is administered once a week, once every two weeks, once every four weeks or once every eight weeks.

11: The method of claim 1, wherein said antibody or antibody fragment is administered at a dose sufficient to achieve a therapeutically effective serum level.

12: The method of claim 11, wherein said serum level is maintained during the treatment course.

13: The method of claim 1, wherein said use is the treatment of moderate-to-severe atopic dermatitis.

14: The method of claim 1, wherein the treatment results in an improvement in an atopic dermatitis-associated parameter selected from the group consisting of:
(a) a decrease from baseline in Eczema Area and Severity Index (EASI) score of at least 55%;
(b) a decrease from baseline in SCORAD score of at least 40%;
(c) a decrease from baseline in Investigator's Global Assessment (IGA) score of at least 40%.

15: The method of claim 14, wherein said improvement in the decrease from baseline in Eczema Area and Severity Index (EASI) score of at least 55% is maintained for at least 4 weeks after the last administered dose.

16: A pharmaceutical composition comprising an antibody or antibody fragment comprising a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15 for use in the treatment of atopic dermatitis, wherein said antibody or antibody fragment is administered at a dose of 0.1 mg/kg-5 mg/kg.

17: The pharmaceutical composition of claim 16 formulated for intravenous or subcutaneous administration.

18: The pharmaceutical composition of claim 16 formulated for subcutaneous administration at a dose of 100 mg-400 mg.

19: The pharmaceutical composition of claim 16 formulated for administration at a dose sufficient to achieve a therapeutically effective serum level.
